(19) United States
(12) Patent Application Publication
(10) Pub. No.: US 2012/0219944 A1
(43) Pub. Date: Aug. 30, 2012

(54) MYH14 AS CAUSATIVE GENE RESPONSIBLE FOR COMPLEX PHENOTYPE OF PERIPHERAL NEUROPATHY, MYOPATHY, HEARING LOSS AND HOARSENESS, AND DIAGNOSTIC METHOD AND KIT FOR THE COMPLEX PHENOTYPE USING THE SAME

(75) Inventors: Byung-Ok CHOI, Seoul (KR); Ki Wha CHUNG, Gongju-si (KR)

(73) Assignees: KONGJU NATIONAL UNIVERSITY INDUSTRY-UNIVERSITY COOPERTION FOUNDATION, Gongju-si (KR); EWHA UNIVERSITY - INDUSTRY COLLABORATION FOUNDATION, Seoul (KR)

(21) Appl. No.: 13/359,389

(22) Filed: Jan. 26, 2012

(30) Foreign Application Priority Data
Feb. 24, 2011 (KR) 10-2011-0016760
Jun. 29, 2011 (KR) 10-2011-0063871

(51) Int. Cl. C12Q 1/68; C40B 40/06; G01N 33/566; C07H 21/04; C07K 2/00

(52) U.S. Cl. 435/6.11; 536/23.5; 530/350; 506/13; 506/16; 436/501; 435/7.92

(57) ABSTRACT

The present invention newly identified a missense mutation in the MYH14 gene as a cause responsible for a complex phenotype of peripheral neuropathy, myopathy, hearing loss, and hoarseness. Further, the present invention provides a method for diagnosing inherited neuromuscular disorders showing a complex phenotype of peripheral neuropathy, myopathy, hearing loss, and hoarseness via detection of the mutated MYH14 gene or the protein encoded thereby, and a diagnostic kit therefor. According to the present invention, simple examination of the gene allows early diagnosis of inherited neuromuscular disorders showing the complex phenotype of peripheral neuropathy, myopathy, hearing loss, and hoarseness, which shows high inheritance and is caused by a single gene defect, and accurate diagnosis of the disease makes it possible to tailor therapy.

### CROSS REFERENCE TO RELATED APPLICATIONS

This application claims benefit from Korean application Nos. 10-2011-0016760 filed on Feb. 24, 2011, and 10-2011-0063871 filed on Jun. 29, 2011, which are incorporated herein by reference in their entirety. This application was supported by a grant of the Korean Health Technology R&D Project, Ministry of Health & Welfare, Republic of Korea (PGM21 Project, A111218-11-GM07).

### STATEMENT REGARDING SEQUENCE LISTING

The Sequence Listing associated with this application is provided in text format in lieu of a paper copy, and is hereby incorporated by reference into the specification. The name of the text file containing the Sequence Listing is HANO_{—}007_{—}00US_ST25.txt. The text file is 43 KB, was created on Jan. 26, 2012, and is being submitted electronically via EFS-Web.

### FIELD OF THE INVENTION

The present invention relates to a mutated MYH14 gene as a causative gene responsible for inherited neuromuscular disorders showing a complex phenotype of peripheral neuropathy, myopathy, hearing loss and hoarseness, and a diagnostic method and a diagnostic kit for the inherited neuromuscular disorders using the same. Specifically, the present invention demonstrates that a novel missense mutation in MYH14 gene, which encodes the nonmuscle myosin heavy chain 14 protein, is a cause responsible for the inherited neuromuscular disorders showing a complex phenotype of peripheral neuropathy, myopathy, hearing loss, and hoarseness. The present invention relates to a method for diagnosing inherited neuromuscular disorders showing a complex phenotype of peripheral neuropathy, myopathy, hearing loss, and hoarseness via detection of the mutated MYH14 gene, and a diagnostic kit therefor.

### BACKGROUND

Hereditary motor and sensory neuropathy, which is also called Charcot-Marie-Tooth disease (CMT), is a genetically and clinically heterogeneous disorder, and exhibits clinical symptoms of distal muscle weakness and atrophy, pes caus, and impaired sensation (Reilly M M, et al., J Neurol Neurosurg Psychiatry 80: 1304-1314, 2009). Autosomal dominant CMT is clinically divided into two types; the demyelinating form (CMT1) and the axonal form (CMT2) (Pareyson D, et al., Lancet Neurol 8: 654-667, 2002). CMT1 has severely reduced median motor nerve conduction velocities (NCVs) (<38 m/sec), whereas CMT2 has slightly reduced or normal NCVs with decreased amplitudes (Harding A E, et al., Brain 103: 259-280, 1980). To date, more than 40 causative genes or loci have been reported to be associated with CMT at the Inherited Peripheral Neuropathies Mutation Database (IPNMD) (http://www.molgen.ua.ac.be/CMTMutations/Mutations/).

Distal myopathies are also clinically and genetically heterogeneous inherited disorders with more than 10 genes and chromosomal loci identified to date (Udd B, Neuromusc Disord 19: 429-438, 2009). A major symptom of distal myopathies is weakness of skeletal muscles in the lower legs and hands occasionally concurrent with cardiomyopathy and vocal dysfunction (Malicdan M C, et al., Neurol India 56: 314-324, 2008]. Autosomal dominant distal myopathies include tibial muscular dystrophy (TMD, MIM #600334) caused by mutations in TTN (MIM #188849) (Hackman P, et al., Am J Hum Genet 71: 492-500, 2002), Laing distal myopathy due to changes in MYH7 (MIM #160760) (Meredith C, et al., Am J Hum Genet 75: 703-708, 2004), distal LGMDIC (caveolinopathy) associated with CAV3 (MIM #6012530) mutations (Minetti C, et al., Nat Genet 18: 365-368, 1998), and myofibrillar myopathies (MFM) caused by mutations in CRYAB (MIM #123590), MYOT (MIM #6041030) and LDB3/ZASP (MIM #605906) (Vicart P, et al., Nat Genet 20: 92-95, 1998; Selcen D, et al., Ann Neurol 57: 269-276, 2005). MATR3 (MIM #164015) have recently been reported as the underlying causes of dominant distal myopathy (Senderek J, et al., Am J Hum Genet 84: 511-518, 2009). Desminopathy and other forms of myofibrillar myopathy are caused by mutations in the DES gene (MIM #125660) with both autosomal dominant and recessive inheritance (Goldfarb L G, et al., Brain 127: 723-734, 2004; Arias M, et al., Neuromuscul Disord 16: 498-503, 2006).

Several patients have been reported to have an unusual incidental combination of two neuromuscular diseases of CMT and muscle diseases, such as myotonic dystrophy, Becker muscular dystrophy (MIM #300376), and facioscapulohumeral muscular dystrophy (BCM, et al., Muscle Nerve 21: 788-791, 1998; Bergmann C, et al., Muscle Nerve 23: 818-823, 2000; Hodapp J A, et al., Arch Neurol 63: 112-117, 2006; Kim H S, et al., Neurogenetics 11: 425-433, 2010). Recently, a Dutch CMT neuropathy family that also showed complex phenotypes of myotonic dystrophy, encephalopathic attacks, and hearing loss revealed an atypical complex mutations at the DM1 locus (MIM #160900) (Spaans F, et al., J Neurol Neurosurg Psychiatry 80: 1029-1035, 2009; Braida C, et al., Hum Mol Genet 19: 1399-1412, 2010). Although many causative genes have been reported to be associated with CMT or distal myopathies, lots of patients are still waiting to uncover their genetic causes. In particular, there has not been reported a mutation that causes diseases showing a complex phenotype of autosomal dominant peripheral neuropathy, distal myopathy, hoarseness, and hearing loss.

Therefore, the present inventors have made an effort to identify the causes of inherited neuromuscular disorders showing this complex phenotype. As a result, they found that a substitution of guanine by thymine at nucleotide position 2822 of the nonmuscle myosin heavy chain 14 (MYH14) gene is a mutation that causes the complex phenotype of peripheral neuropathy, distal myopathy, hoarseness, and hearing loss, and the mutated MYH14 gene can be used for the diagnosis of inherited neuromuscular disorders showing the complex phenotype, thereby completing the present invention.

### SUMMARY OF THE INVENTION

An object of the present invention is to provide a mutated MYH14 gene having a substitution of guanine by thymine at position 2822 of the base sequence of SEQ ID NO: 1 as a diagnostic marker for inherited neuromuscular disorders showing a complex phenotype of peripheral neuropathy, myopathy, hearing loss, and hoarseness.

Another object of the present invention is to provide a mutated Myh14 protein encoded by the mutated MYH14 gene.

Still another object of the present invention is to provide a diagnostic composition for inherited neuromuscular disorders showing a complex phenotype of peripheral neuropathy, myopathy, hearing loss, and hoarseness, comprising an agent capable of detecting the expression of mRNA of the mutated MYH14 gene or a mutated Myh14 protein encoded by the gene in a sample of an individual.

Still another object of the present invention is to provide a diagnostic kit for inherited neuromuscular disorders showing a complex phenotype of peripheral neuropathy, myopathy, hearing loss, and hoarseness, comprising the composition.

Still another object of the present invention is to provide a method for diagnosing inherited neuromuscular disorders showing a complex phenotype of peripheral neuropathy, myopathy, hearing loss, and hoarseness, comprising the step of detecting the expression of mRNA of the mutated MYH14 gene or a mutated Myh14 protein encoded by the gene.

### BRIEF DESCRIPTION OF THE DRAWINGS

FIG. 1 is the pedigree of the family FC317 showing a complex phenotype of peripheral neuropathy, myopathy, hearing loss, and hoarseness as a subject of the present invention, in which haplotypes (indicated below each familial member) were determined using genotypes of microsatellite markers at chromosome 19q13.3 (genotypes in parentheses were inferred), and * indicates individuals whose DNA was used for linkage analysis, black color indicates phenotypes confirmed by clinical exam, gray indicates presumptive phenotypes based on history talking, and [symbol] indicates the proband;

FIG. 2 is photographs of patients with distal muscle weakness of the lower limbs, showing that the progressive leg muscle atrophy was associated with disease duration (DD) (A: DD=4 years (F/11, IV-14); B: DD=28 years (F/41, III-13); C: DD=38 years (M/48, III-8); D: DD=41 years (M/52, III-6));

FIGS. 3a to 3e show linkage analysis of family FC317 according to the present invention and identification of a missense mutation in MYH14 gene, in which FIG. 3a shows two-point LOD scores of chromosome 19, FIG. 3b shows chromosomal fine mapping of the 19q13.2-3 region and genotyping of 29 microsatellites revealed a ˜13 Mb linkage region flanked by markers D19S902 and D19S246, FIG. 3c shows a diagram of chromosome 19q13.2-3 and markers that cosegregate with the phenotype are indicated in bold, FIG. 3d shows sequencing chromatograms of the c.2822G>T(Arg941Leu) mutation in the MYH14 gene and the variant cosegregated with all the affected members in family FC317, and FIG. 3e shows high conservation of the arginine residue at position 941 illustrated by alignment of the amino acid sequences of Myh14 protein family from different species (H. sapiens Myh14-NP_{—}001070654.1; M. musculus Myh14-NP_{—}082297.1; R. norvegicus Myh14-NP_{—}001094160.1; B. Taurus Myh14-XM_{—}882711.4) using a ClustalX program (ver. 1.83);

FIG. 4a shows a Log₂ ratio plot obtained by CGH microarray analysis, in which two affected patients, 1 unaffected patient and CMT1A and HNPP patients as each control group were subjected to the analysis, and the CGH microarray analysis revealed no CNV within the linkage disequilibrium region (36.6-60.2 Mb, hg18);

FIG. 4b is the result of karyotyping of male and female patients, showing no chromosomal abnormality (Upper portion: male patient III-16, Lower portion: female patient III-13);

FIG. 5 is the result of showing MYH14 expression in the gastrocnemius muscle, in which the expression level in biopsied tissue was determined by quantitative real-time PCR and GAPDH expression was used as a control, and CTL-Vl expression level was determined as 1.0 (CTL-Vl: vastus lateralis of healthy male, CTL-Ga: gastrocnemius muscles of healthy male, and III-16-Ga: gastrocnemius muscles of III-16 male patient);

FIG. 6 shows motor unit action potential (MUAP), in which A shows the interference patterns in patient III-8 made from the right vastus lateralis, and B shows the interference patterns in patient III-8 made from the first dorsal interosseous, the patient has evidence, clinically and electrophysiologically, of both neuropathy and myopathy, and the electrophisiological studies revealed both a large amplitude with long duration (neuropathic, A) and a small amplitude with short duration polyphasic (myopathic) motor units (B);

FIG. 7 shows a sequential pattern of muscle involvement associated with disease duration (DD) in the T1-weighted axial MRI, in which A: a 16-year-old male patient (IV-10, DD=4 years) showed mild streaky fatty infiltrations of the anterior compartment muscles of the legs, B: a 15-year-old male patient (IV-13, DD=5 years) displayed more fatty infiltrations, including the anterior and the lateral compartments, C: a 33-year-old male (III-16, DD=24 years) showed prominent involvement of the anterior and lateral compartments of leg muscles with mild involvement of the posterior compartment, D: a 41-year-old female patient (III-13, DD=28 years) showed severe fatty involvement of all muscles compartments including a calf muscle atrophy (anterior compartment: blank arrowheads, lateral compartment: arrowheads, posterior compartment: arrow); and

FIG. 8 shows histopathologic findings of lateral gastrocnemius muscle in patient III-16, in which A shows marked variation of fiber size and shape with many small rounded fibers and degenerating fibers, and endomysial fibrosis was prominent on the background of degenerating myofibers without inflammatory cells (Magnification: ×200), B shows grouping of histochemical fiber types, and ATPase reaction with different pH preincubation and immunostaining with myosin heavy chain (fast), myosin heavy chain (slow), and myosin IIa showed grouping of histochemical fiber types (Magnification: ×40), and C shows subsarcolemmal accumulation of abnormal mitochondria, and electron micrographs revealed subsarcolemmal accumulation of frequent abnormal mitochondria including variable sized rectangular or elongated rhomboidal paracrystalline inclusions (Magnification: ×30,000).

### DETAILED DESCRIPTION OF THE INVENTION

In one embodiment, the present invention provides a missense mutation in the MYH14 gene, which encodes the nonmuscle myosin heavy chain 14 protein, as a causative gene responsible for inherited neuromuscular disorders showing a complex phenotype of peripheral neuropathy, myopathy, hearing loss, and hoarseness.

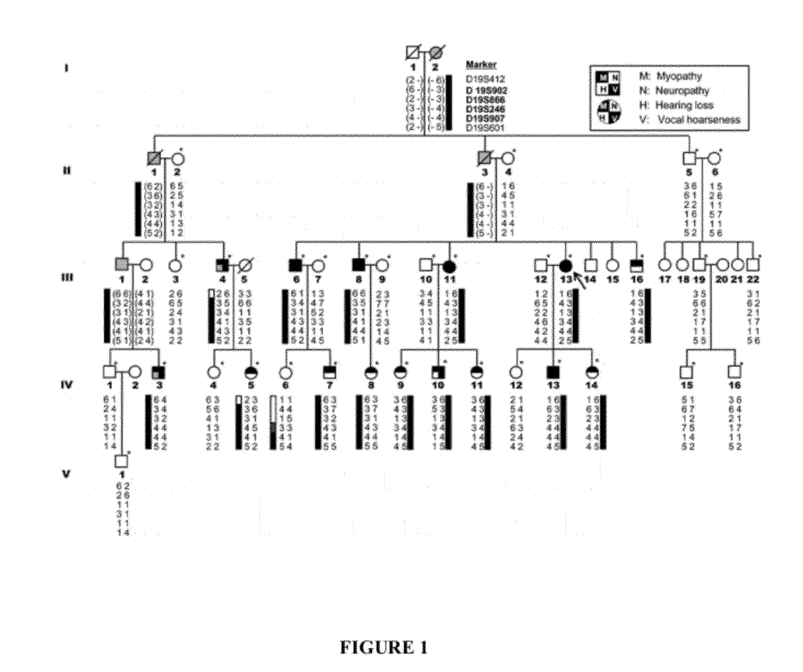

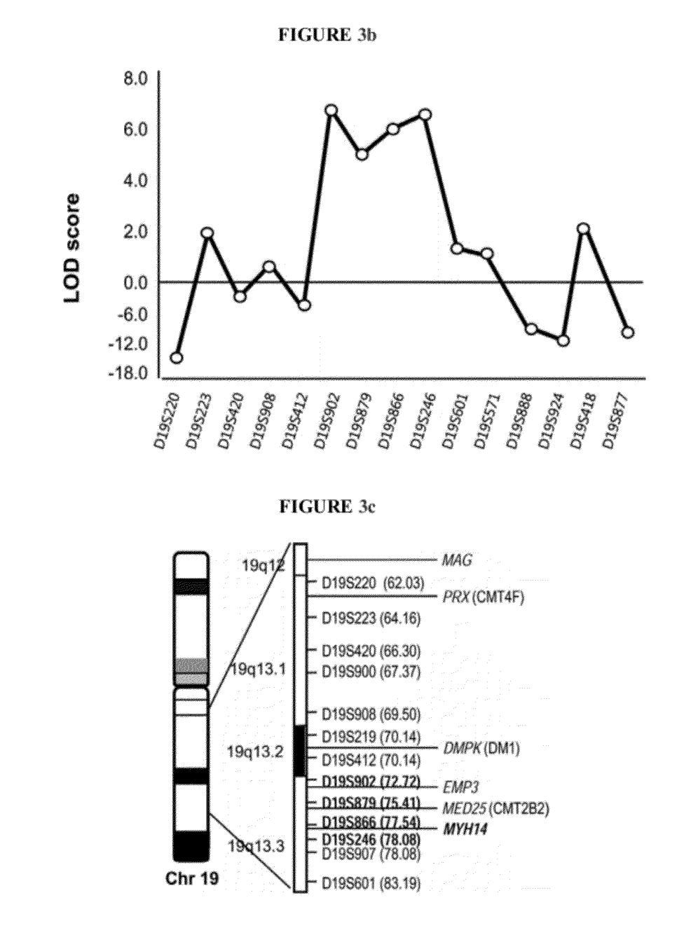

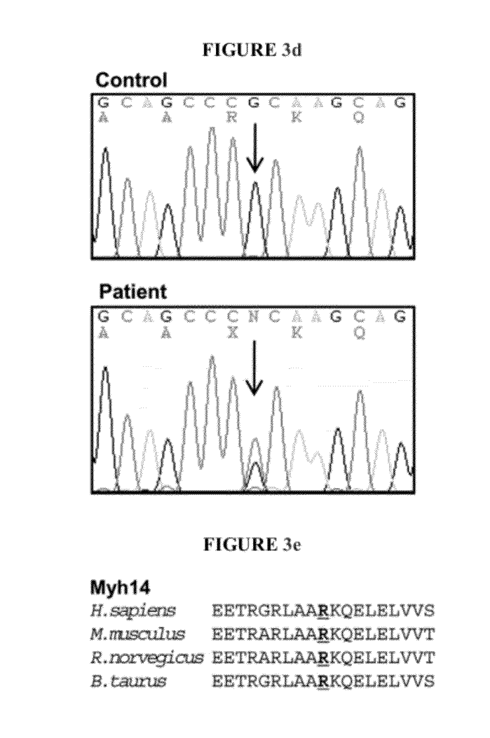

In the present invention, the present inventors have mapped a new chromosomal locus for the complex phenotype to 19q13.3 in a large autosomal dominant family with the complex phenotype of peripheral neuropathy, distal myopathy, hoarseness, and hearing loss (see FIGS. 1 and 3b). Sequencing analysis of 34 candidate genes revealed a novel missense mutation in the gene MYH14 encoding the nonmuscle myosin heavy chain 14 protein that is specifically detected in patients with the complex phenotype (see FIG. 3d).

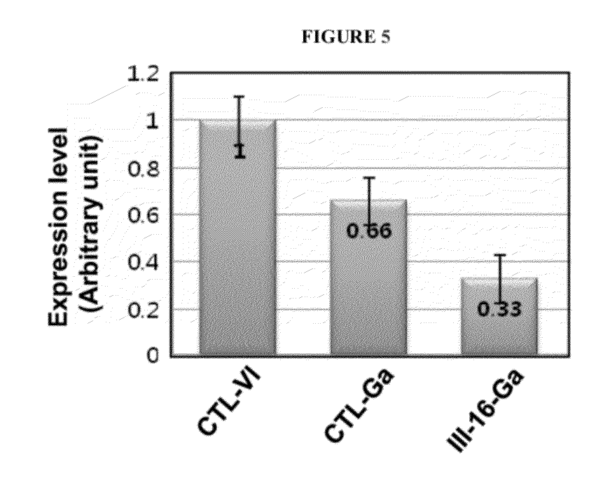

MYH14 gene (NM_{—}001077186.1) consists of 41 exons (40 coding), which comprise 108 kbp of genomic sequence and has a base sequence represented by SEQ ID NO: 1. The gene encodes the Myh14 protein consisting of 2003 amino acids, which has an amino acid sequence represented by SEQ ID NO: 2. The MYH14 gene encodes four conserved functional domains: the amino-terminal myosin domain, the myosin head, two IQ domains, and the myosin tail. Strong expression of the 7 kbp transcript of the MYH14 gene was found in the skeletal muscle, small intestine, colon, and cochlea, but it is also expressed in a wide range of tissue including brain and peripheral nerves (Leal A, et al., Gene 312: 165-171, 2003; Donaudy F, et al., Am J Hum Genet 74: 770-776, 2004; Golomb E, et al., J Biol Chem 279: 2800-2808, 2004). In the present invention, MYH14 expression was first identified in the gastrocnemius muscle (see FIG. 5).

According to the present invention, a missense mutation (2822G>T) of a substitution of guanine by thymine at nucleotide position 2822 of the MYH14 gene having the base sequence of SEQ ID NO: 1 was specifically identified in the patients with the complex phenotype of peripheral neuropathy, distal myopathy, hoarseness, and hearing loss. The Myh14 protein encoded by the MYH14 gene having the mutation includes a mutation showing a substitution of an arginine residue with leucine at amino acid position 941 of SEQ ID NO: 2 (Arg941Leu).

The present invention supports that the 2822G>T mutation in the MYH14 gene is a causative gene responsible for inherited neuromuscular disorders showing the complex phenotype of peripheral neuropathy, myopathy, hoarseness, and hearing loss due to the following reasons: (1) co-segregation of the mutation with affected members in the pedigree, (2) no detection of the same mutation in 566 ethnicity matched control chromosomes, (3) high conservation of amino acids at the mutation site among different species, (4) previous involvement of MYH14 in nonsyndromic hearing loss, and (5) absence of alternative causative mutations in known CMT or distal myopathy genes.

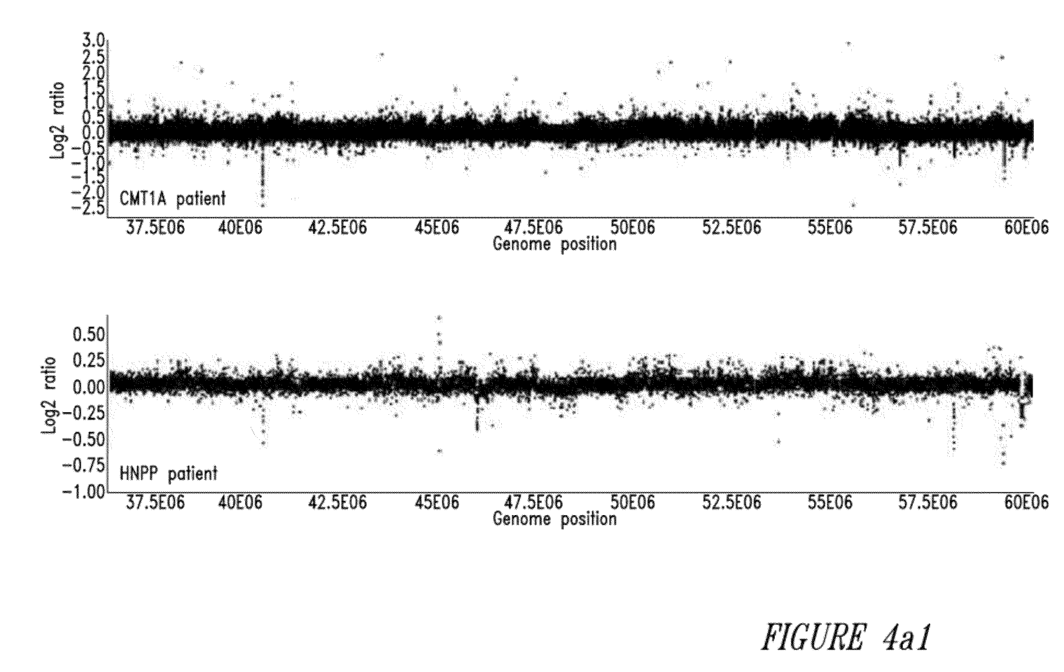

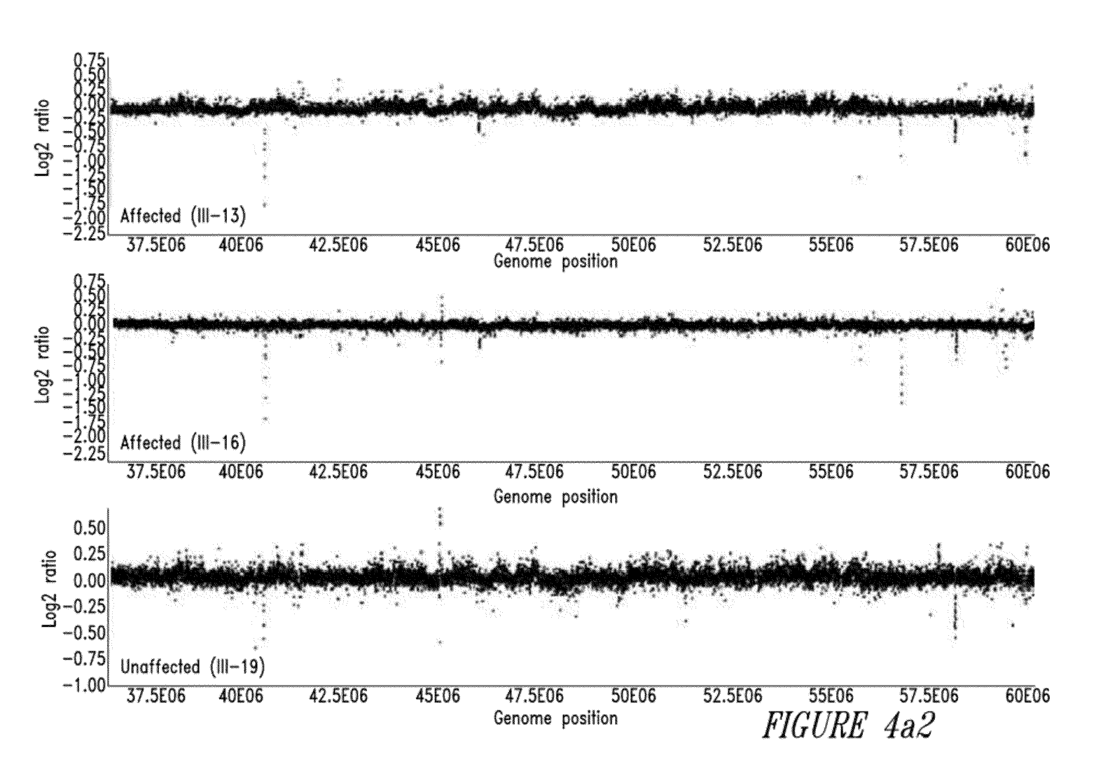

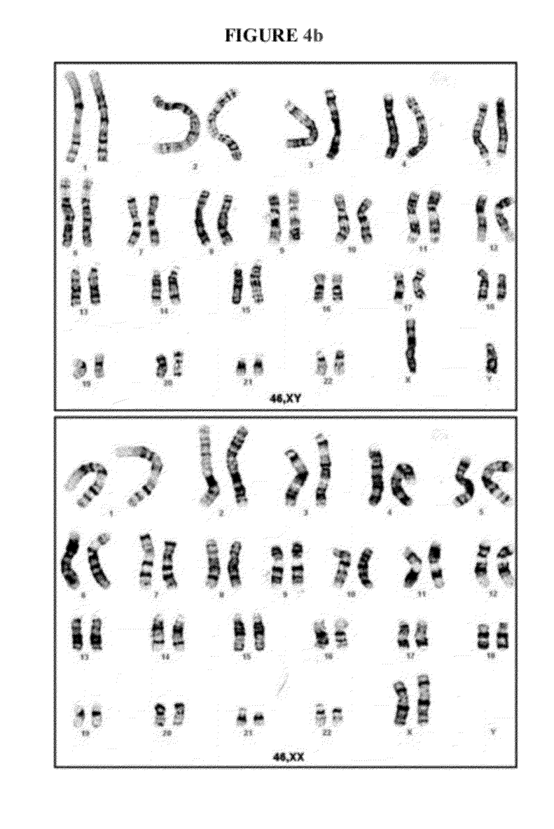

Moreover, CNV (copy-number variations) examination revealed no significant duplication or deletion in the linkage region (see FIG. 4a). Karyotyping also showed no chromosomal abnormality (see FIG. 4b). Recently, complex mutations in the DMPK gene were reported as the underlying cause of a sensorimotor neuropathy family with myotonic dystrophy, encephalopathic attacks, and hearing loss (Spaans F, et al., J Neurol Neurosurg Psychiatry 80: 1029-1035, 2009; Braida C, et al., Hum Mol Genet 19: 1399-1412, 2010). However, the present inventors excluded mutations in DMPK, which falls near 19q13.3 and also sequenced additional candidate genes, including PRX (CMT4F), MED25 (CMT2B2), MAG, and EMP3, but they did not identify significant nucleotide changes. The complex phenotype of the Dutch family is in some extent similar to the Korean family according to the present invention. However, no myotonic discharges were found in the family of the present invention.

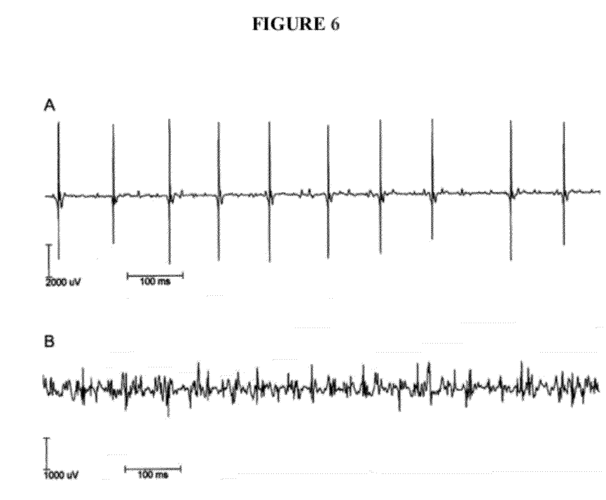

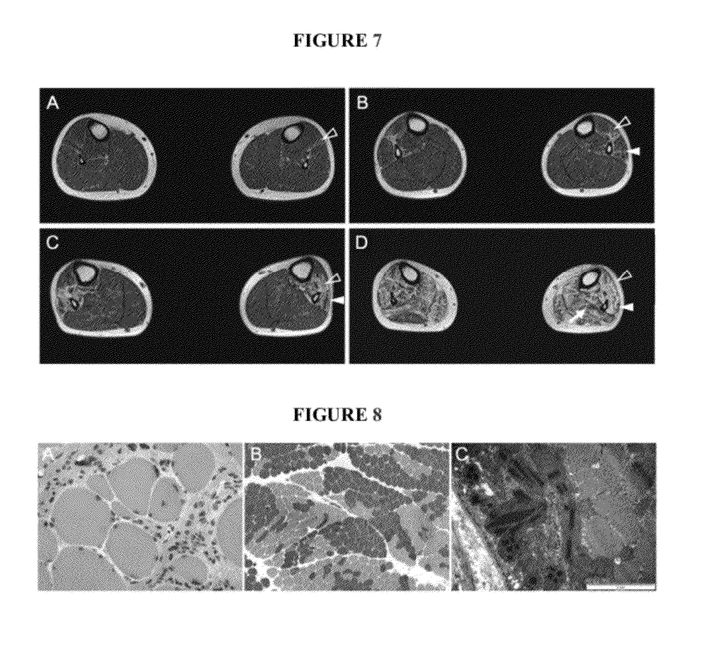

Electrophysiological study showed both chronic neuropathic and myopathic features in the affected patients (see Table 4). Electromyography (EMG) results showed a small amplitude short duration myopathic MUAPs, and also revealed neuropathic MUAPs in the same affected individuals (see FIG. 6). In addition, MRI results showed a sequential pattern of muscle involvement associated with disease duration (see FIG. 7).

Histopathologic findings revealed marked variation of fiber size with many small round or angulated fibers, degenerating fibers, and endomyseal fibrosis, but also showed grouping of histochemical muscle fiber types, which was one of the well-known features noted in neurogenic changes of skeletal muscle (see FIG. 8). Additionally, variable sized rectangular or rhomboidal paracrystalline inclusions were frequently found in electron micrographs. Because the histopathologic abnormalities were reminiscent of cases with mitochondrial diseases, the present inventors completely sequenced the mitochondrial DNA from two biopsied patients (III-6 and III-13), but found no causative mutation (see Table 2). The histological study suggested that the pathogenic mechanism of inherited neuromuscular disorders may be related with abnormal translocation and dysfunction of mitochondria.

Myosins are a superfamily with a domain that interacts with actin in order to produce movement under hydrolysis of ATP. The three known nonmuscle myosins are encoded by the MYH9 (MIM #160775), MYH10 (MIM #160776) and MYH14 genes located on chromosomes 22q11.2, 17p13.3 and 19q13.3, respectively (Simons M, et al., Circulation Res 69: 530-539, 1991; Leal A, et al., Gene 312: 165-171, 2003). Myosin heavy chain genes underlie several forms of hereditary hearing loss. The hearing loss loci, DFNB2 (MIM #600060) and DFNA17 (MIM #600652), are associated with MYH7A (MIM #160760) (Astuto L M, et al., Am J Med Genet 109: 291-297, 2002) and MYH9 mutation (Lalwani A K, et al., Am J Hum Genet 67: 1121-1128, 2000), respectively. The nonsyndromic autosomal dominant form of hearing impairment, DFNA4 (MIM #600652), is caused by mutations in MYH14 (Mirghomizadeh F, et al., Eur J Hum Genet 10: 95-99, 2002; Donaudy F, et al., Am J Hum Genet 74: 770-776, 2004). In contrast, the Korean family studied in the present invention exhibited a syndromic phenotype, dominated by peripheral neuropathy, myopathy, hoarseness, and hearing loss. Therefore, it is suggested that the present invention significantly extents the known phenotype associated with MYH14 mutations.

In conclusion, the mutated gene newly identified in the present invention as a causative gene responsible for inherited neuromuscular disorders showing the complex phenotype of peripheral neuropathy, myopathy, hoarseness and hearing loss is as follows:

MYH14 c.2282G>T: substitution of guanine with thymine at position 2282 of the base sequence of the MYH14 gene represented by SEQ ID NO: 1, leading to a substitution of an arginine residue with leucine at position 941 of the amino acid sequence represented by SEQ ID NO: 2.

As a diagnostic marker for inherited neuromuscular disorders showing the complex phenotype of peripheral neuropathy, myopathy, hearing loss, and hoarseness, therefore, the present invention provides the mutated MYH14 gene having a substitution of guanine with thymine at position 2282 of the base sequence represented by SEQ ID NO: 1, and the mutated Myh14 protein encoded by the mutated MYH14 gene.

The mutated MYH14 gene and/or the mutated Myh14 protein encoded thereby according to the present invention can be effectively used for diagnosing the incidence of inherited neuromuscular disorders showing the complex phenotype of peripheral neuropathy, myopathy, hearing loss, and hoarseness.

The term “diagnosis”, as used herein, refers to evaluation of the presence or properties of pathological states. With respect to the objects of the present invention, the diagnosis is to determine the incidence of inherited neuromuscular disorders showing a complex phenotype of peripheral neuropathy, myopathy, hearing loss, and hoarseness.

The term “diagnostic marker”, as used herein, is a substance capable of diagnosing the incidence of inherited neuromuscular disorders showing the complex phenotype. With respect to the objects of the present invention, the diagnostic marker is the mutated MYH14 gene according to the present invention, which is specifically detected in patients showing the complex phenotype, unlike normal persons.

The selection and application of significant diagnostic markers determine the reliability of diagnosis results. A significant diagnostic marker means a marker that has high validity, giving accurate diagnosis results, and high reliability, supplying constant results upon repeated measurement. The mutated MYH14 gene according to the present invention as a diagnostic marker is a marker having high reliability, which is detected only in patients showing the complex phenotype of peripheral neuropathy, myopathy, hearing loss, and hoarseness, and rarely detected in a normal control group. Therefore, diagnosis based on the results obtained by detecting the presence of the mutated MYH14 gene of the present invention as a significant diagnostic marker is valid and reliable.

In another embodiment of the present invention, the present invention relates to a diagnostic composition for inherited neuromuscular disorders showing a complex phenotype of peripheral neuropathy, myopathy, hearing loss, and hoarseness, comprising an agent capable of detecting the presence of the mutated MYH14 gene or the mutated Myh14 protein encoded thereby in a sample of an individual.

In the diagnostic composition according to the present invention, the agent for detecting the presence of the mutated MYH14 gene may be a primer or probe that is designed to detect the substitution of guanine with thymine at position 2282 of the base sequence of the MYH14 gene represented by SEQ ID NO: 1. A primer or probe capable of specifically amplifying the specific region of the gene can be designed on the basis of the base sequence of the MYH14 gene. The base sequence of the MYH14 gene is currently available in GenBank and is known in the art, and the mutated region used as a diagnostic marker for inherited neuromuscular disorders showing the complex phenotype of peripheral neuropathy, myopathy, hearing loss, and hoarseness was also identified by the present inventors. Therefore, the primer or probe capable of specifically amplifying the specific region of the mutated MYH14 gene can be easily designed by those skilled in the art, based on the above base sequence.

The term “primer”, as used herein, means a short nucleic acid sequence having a free 3′-hydroxyl group, and a single strand oligonucleotide which is able to form base-pairing interaction with a complementary template and serves as a starting point for'replication of the template strand. A primer is able to act as a point of initiation of template-directed DNA synthesis under suitable conditions (e.g., in the presence of four different nucleoside triphosphates and an agent for polymerization, such as DNA or RNA polymerase or reverse transcriptase) in an appropriate buffer and at a suitable temperature. The suitable length of a primer will depend on its intended use, but typically ranges from 15 to 30 nucleotides. A short primer molecule generally requires a lower temperature to be stably hybridized with the template. The primer sequence does not necessarily need to be completely complementary to the template, but should be sufficiently complementary to be hybridized with the template. In the present invention, forward and reverse primers for the mutated MYH14 gene are used to perform PCR amplification, and then amplification of the PCR product is examined to diagnose the incidence of inherited neuromuscular disorders showing the complex phenotype of peripheral neuropathy, myopathy, hearing loss, and hoarseness.

The suitable primer in the present invention is a primer that is specific to the mutated MYH14 gene, namely, that is designed to detect a substitution of guanine by thymine at nucleotide position 2822 of the MYH14 gene of SEQ ID NO: 1, and is sense and antisense oligonucleotides having 7 to 50 nucleotide sequences.

The term “probe”, as used herein, refers to a nucleic acid (e.g., DNA or RNA) fragment capable of specifically binding to mRNA, ranging in length from a single base to hundreds of bases. The probe may be prepared in the form of oligonucleotide probes, single stranded DNA probes, double stranded DNA probes, or RNA probes. In the present invention, hybridization is performed using a probe complementary to the mutated MYH14 gene, and inherited neuromuscular disorders showing the complex phenotype of peripheral neuropathy, myopathy, hearing loss, and hoarseness can be diagnosed by the hybridization result. Selection of suitable probe and hybridization conditions can be modified on the basis of the methods known in the art.

The primer or probe according to the present invention may be chemically synthesized using a phosphoramidite solid support method or other widely known methods. These nucleic acid sequences may also be modified using many means known in the art. Non-limiting examples of such modifications include methylation, capsulation, replacement of one or more native nucleotides with analogues thereof, and inter-nucleotide modifications, for example, modifications to uncharged conjugates (e.g., methyl phosphonate, phosphotriester, phosphoroamidate, carbamate, etc.) or charged conjugates (e.g., phosphorothioate, phosphorodithioate, etc.).

In accordance with a preferred embodiment of the present invention, the diagnostic composition includes a pair of primers represented by SEQ ID NOs: 51 and 52, which are designed to specifically hybridize with exon 3 of the MYH14 gene, and to detect a substitution of guanine with thymine at position 2282 of the base sequence represented by SEQ ID NO: 1.

The presence of the mutated MYH14 gene in a sample of an individual can be examined by assessing the expression of mRNA or protein of the gene.

The “measurement of mRNA expression”, as used herein, is a process of assessing the presence and expression levels of mRNA of the mutated MYH14 gene in a sample of an individual, which can be assessed by measuring the amount of mRNA. The analysis methods include, but are not limited to, reverse transcription polymerase chain reaction (RT-PCR), competitive reverse transcription polymerase chain reaction (competitive RT-PCR), real-time reverse transcription polymerase chain reaction (real-time RT-PCR), RNase protection assay (RPA), Northern blotting and DNA chip assay.

The “measurement of protein expression”, as used herein, is a process of assessing the presence and expression levels of a mutated Myh14 protein expressed from the mutated MYH14 gene in a sample of an individual. Preferably, the amount of protein product can be measured using antibodies specifically binding to the protein encoded by the gene. The analysis methods include, but are not limited to, Western blotting, enzyme linked immunosorbent assay (ELISA), radioimmunoassay (RIA), radial immunodiffusion, Ouchterlony immunodiffusion, rocket immunoelectrophoresis, immunohistostaining, immunoprecipitation assay, complement fixation assay, Fluorescence Activated Cell Sorter (FACS), and protein chip assay.

The term “antibody”, as used herein, is a term known in the art, and refers to a specific protein molecule that indicates an antigenic region. With respect to the objects of the present invention, the antibody refers to an antibody that specifically binds to the mutated Myh14 protein encoded by the mutated MYH14 gene of the present invention. To prepare the antibody, the mutated MYH14 gene is cloned into an expression vector according to the typical method, so as to obtain a protein encoded by the gene, and then the antibody may be prepared from the obtained protein according to the typical method, in which a partial peptide prepared from the protein is also included, and the partial peptide of the present invention includes at least 7 amino acids, preferably 9 amino acids, and more preferably 12 amino acids or more. There is no limitation in the form of the antibody of the present invention, and a polyclonal antibody, a monoclonal antibody, or a part thereof having antigen-binding property is also included, and all immunoglobulin antibodies are included.

The MYH14 gene and its mutated region were identified as a diagnostic marker for inherited neuromuscular disorders showing the complex phenotype of peripheral neuropathy, myopathy, hearing loss, and hoarseness by the present invention, and thus antibody production using the same may be easily carried out using techniques widely known in the art.

Polyclonal antibodies may be produced by a method widely known in the art, which includes injecting the protein antigen encoded by the mutated MYH14 gene into an animal and collecting blood samples from the animal to obtain serum containing antibodies. Such polyclonal antibodies may be prepared from a certain animal host, such as goats, rabbits, sheep, monkeys, horses, pigs, cows and dogs.

Monoclonal antibodies may be prepared by a method widely known in the art, such as a hybridoma method (Kohler and Milstein, European Journal of Immunology 6: 511-519, 1976), or a phage antibody library technique (Clackson et al, Nature 352: 624-628, 1991; Marks et al, J Mol Biol 222(58): 1-597, 1991). Antibodies prepared by the above methods are isolated and purified using gel electrophoresis, dialysis, salting out, ion exchange chromatography, affinity chromatography, and the like.

Furthermore, the antibody of the present invention also includes recombinant antibodies, such as a humanized antibody. The antibodies used in the present invention include complete forms having two full-length light chains and two full-length heavy chains, as well as functional fragments of antibody molecules. The functional fragments of antibody molecules refer to fragments retaining at least an antigen-binding function, and include Fab, F(ab′), F(ab′)₂, Fv and the like.

In still another embodiment, the present invention provides a kit for diagnosing the incidence of inherited neuromuscular disorders showing the complex phenotype of peripheral neuropathy, myopathy, hearing loss, and hoarseness, comprising the diagnostic composition.

The kit of the present invention is used to diagnose the incidence of inherited neuromuscular disorders showing the complex phenotype of peripheral neuropathy, myopathy, hearing loss, and hoarseness by determining mRNA expression of the mutated MYH14 gene or expression of the mutated Myh14 protein encoded by the gene in a subject and then examining c.2282G>T mutation in the MYH14 gene or p.Arg941Leu mutation in the Myh14 protein.

The kit of the present invention may include a primer or probe to detect the mRNA expression of the mutated MYH14 gene, and an Antibody selectively recognizing the mutated Myh14 protein encoded by the gene, as well as one or more kinds of a composition, a solution, or an apparatus, which are suitable for the analysis method.

In a specific embodiment, the kit to measure the mRNA expression level of the mutated MYH14 gene may be a kit characterized by the inclusion of essential elements required for performing RT-PCR. An RT-PCR kit may include test tubes or other suitable containers, reaction buffers, deoxynucleotides (dNTPs), enzymes such as Taq-polymerase and reverse transcriptase, DNase, RNase inhibitor, DEPC water, and sterile water, in addition to a pair of primers specific for the mutated MYH14 gene.

Further, the kit of the present invention may be in the form of a microarray including the mutated MYH14 gene according to the present invention. The microarray may include a DNA or RNA polynucleotide probe. The microarray includes the typical microarray constitution except for including a probe specific to the base sequence of the mutated MYH14 gene according to the present invention. The microarray of the present invention may provide information that is useful for diagnosing the incidence of inherited neuromuscular disorders showing the complex phenotype of peripheral neuropathy, myopathy, hearing loss, and hoarseness by detecting the presence of the mutated MYH14 gene according to the present invention.

A method of fabricating the microarray by fixing a probe specific to the mutated MYH14 gene according to the present invention on a substrate is well known in the art. For example, a probe specific to the marker gene according to the present invention may be immobilized onto the substrate using a piezoelectric micropipetting technique or a pin-type spotter, but is not limited thereto. The substrate of the microarray of the present invention is preferably coated with a functional group selected from a group consisting of amino-silane, poly-L-lysine, and aldehyde, but is not limited thereto. The substrate is preferably selected from a group consisting of glass, plastic, metal, silicon, a nylon membrane, and a nitrocellulose membrane, but is not limited to thereto.

In addition, nucleic acid hybridization on the microarray and detection of the hybridization result are well known in the art. The nucleic acid sample is labeled with a fluorescent material, for example, a labeling material capable of producing detectable signals, such as Cy3 and Cy5, and hybridization is performed on the microarray, and the signals produced from the labeling material are detected, thereby detecting the hybridization result.

In the present invention, the kit for measuring the expression level of the protein encoded by the mutated MYH14 gene may include a matrix, a suitable buffer solution, a coloring enzyme, or a secondary antibody labeled with a fluorescent substance, a coloring substrate or the like for the immunological detection of antibody. As for the matrix, a nitrocellulose membrane, a 96 well plate made of polyvinyl resin, a 96 well plate made of polystyrene resin, and a slide glass may be used. As for the coloring enzyme, peroxidase and alkaline phosphatase may be used. As for the fluorescent substance, FITC and RITC may be used, and as for the coloring substrate solution, ABTS (2,2′-azino-bis(3-ethylbenzthiazoline-6-sulfonic acid)), OPD (o-phenylenediamine), or TMB (tetramethyl benzidine) may be used.

In still another embodiment, the present invention relates to a method for diagnosing inherited neuromuscular disorders showing the complex phenotype of peripheral neuropathy, myopathy, hearing loss, and hoarseness, comprising the step of measuring the mRNA expresion of the mutated MYH14 gene or expression of the mutated Myh14 protein encoded by the gene in a subject.

The diagnostic method according to the present invention may include the steps of:

1) measuring mRNA expression of the mutated MYH14 gene or expression of the protein encoded by the gene in a sample of an individual; and

2) determining that the individual has a high risk of inherited neuromuscular disorders showing the complex phenotype of peripheral neuropathy, myopathy, hearing loss, and hoarseness, when the mRNA expression of the mutated MYH14 gene or expression of the protein encoded by the gene is detected in the sample.

The term “a sample of an individual”, as used herein, includes tissues, cells, whole blood, serum, plasma, saliva, sputum, cerebrospinal fluid, or urine that is separated from an individual in order to examine the incidence of inherited neuromuscular disorders showing the complex phenotype of peripheral neuropathy, myopathy, hearing loss, and hoarseness, but is not limited thereto.

Analysis methods for measuring the mRNA expression include, but are not limited to, reverse transcription polymerase chain reaction, competitive reverse transcription polymerase chain reaction, real-time reverse transcription polymerase chain reaction, RNase protection assay, Northern blotting, and DNA chip assay.

With the detection methods, the mRNA expression level of the mutated MYH14 gene can be measured in an individual of having the suspected disease, and the incidence of inherited neuromuscular disorders showing the complex phenotype of peripheral neuropathy, myopathy, hearing loss, and hoarseness can be determined by examining the detection of the mRNA expression. The mRNA expression may be preferably measured by reverse transcription polymerase chain reaction using primers specific to the mutated MYH14 gene as a diagnostic marker or DNA microarray chip using probes specific to the gene.

In a specific embodiment, reverse transcription polymerase chain reaction is performed using primers specific to the mutated MYH14 gene as a diagnostic marker, and then the PCR products are electrophoresed, and patterns and thicknesses of bands are analyzed to determine the mRNA expression of the mutated gene, thereby easily diagnosing the incidence of inherited neuromuscular disorders showing the complex phenotype of peripheral neuropathy, myopathy, hearing loss, and hoarseness.

Meanwhile, as for the DNA microarray chip, it includes the mutated MYH14 gene or a nucleic acid corresponding to its fragment that is very densely arranged on a substrate such as a glass plate. The mRNA isolated from a sample is used to synthesize cDNA probes labeled at an end or at an internal site with a fluorescent material. The cDNA probes are hybridized with the DNA chip, thereby easily diagnosing the incidence of inherited neuromuscular disorders showing the complex phenotype of peripheral neuropathy, myopathy, hearing loss, and hoarseness.

In detail, the analysis method using the DNA microarray chip may include the following steps:

1) isolating mRNAs of the mutated MYH14 gene from a sample of an individual;

2) synthesizing cDNAs from the mRNAs isolated from the sample, with a fluorescent material incorporated thereinto;

3) hybridizing the fluorescent-labeled cDNAs with a DNA microarray chip, where probes specific to the mutated MYH14 gene is immobilized; and

4) analyzing the hybridized DNA microarray chip to detect expression of the mutated MYH14 gene in the sample of the individual.

Examples of the fluorescent materials useful in the above analysis method include, but are not limited to, Cy3, Cy5, FITC (poly L-lysine-fluorescein isothiocyanate), RITC (rhodamine-B-isothiocyanate) and rhodamine. In addition, examples of the DNA microarray chip may include 36 k Human V4.0 OpArray oligomicroarray (Operon, Germany) or whole human genome oligo microarray (Agilent, USA), but are not limited thereto.

Assay methods for measuring the protein expression may be exemplified by Western blotting, ELISA, radioimmunoassay, radioimmunodiffusion, Ouchterlony immunodiffusion, rocket immunoelectrophoresis, histoimmunostaining, immunoprecipitation assay, complement fixation assay, FACS, and protein chip, but are not limited thereto.

By this assay method, the quantities of the formed antigen-antibody complexes can be detected in a subject suspected of having the disease, and expression of the protein encoded by the mutated MYH14 gene can be determined, thereby diagnosing the incidence of inherited neuromuscular disorders showing the complex phenotype of peripheral neuropathy, myopathy, hearing loss, and hoarseness.

The term “antigen-antibody complex”, as used herein, means a conjugate of the protein encoded by the mutated MYH14 gene and an antibody specific thereto. The formation of an antigen-antibody complex may be quantitatively determined by measuring the signal intensity of the detection label.

The measurement of protein expression may be also achieved using ELISA. Examples of ELISA include direct ELISA in which a labeled antibody immobilized onto a solid support is used to recognize an antigen, indirect ELISA in which a labeled antibody is used to recognize a captured antibody immobilized on a solid support which is complexed with an antigen, direct sandwich ELISA in which an antibody is used to recognize an antigen captured by another antibody immobilized onto a solid support, and indirect sandwich ELISA in which a secondary antibody is used to recognize an antibody which captures an antigen complexed with a different antibody immobilized onto a solid support.

Further, the measurement of protein expression may be achieved by Western blotting using one or more antibodies against the protein encoded by the mutated MYH14 gene. Proteins are isolated from a sample, separated according to size by electrophoresis, transferred onto a nitrocellulose membrane, and reacted with an antibody. The quantity of the formed antigen-antibody complex is measured using a labeled antibody to determine an amount of the protein produced by gene expression, thereby diagnosing the incidence of inherited neuromuscular disorders showing the complex phenotype of peripheral neuropathy, myopathy, hearing loss, and hoarseness.

Further, a histoimmunostaining method may be performed using one or more antibodies against the protein encoded by the mutated MYH14 gene. A tissue taken from a subject is fixed and embedded in paraffin according to the method widely known in the art. The paraffin block is cut into slices having a thickness of several and then the slices are placed on glass slides to prepare tissue slices. An antibody against the protein encoded by the mutated MYH14 gene according to the present invention is applied thereto according to the known method, followed by washing off of the unreacted antibodies. Thereafter, the antibody is reacted with a color developing agent, and the protein expression is then observed under a microscope, thereby diagnosing the incidence of inherited neuromuscular disorders showing the complex phenotype of peripheral neuropathy, myopathy, hearing loss, and hoarseness.

Further, a protein chip, in which one or more antibodies against the protein encoded by the mutated MYH14 gene are arranged at predetermined positions and fixed at a high density on a substrate, may be used. In this regard, proteins isolated from a sample are hybridized with the protein chip to form antigen-antibody complexes. The formation of the antigen-antibody complex can be thus quantitatively read so as to examine the presence or expression level of the protein, thereby diagnosing the incidence of inherited neuromuscular disorders showing the complex phenotype of peripheral neuropathy, myopathy, hearing loss, and hoarseness.

As described above, a mutated MYH14 gene was first identified in the present invention as a causative gene responsible for inherited neuromuscular disorders showing a complex phenotype of peripheral neuropathy, myopathy, hearing loss, and hoarseness in Korean patients. Therefore, examination of the mutated MYH14 gene allows early diagnosis of inherited neuromuscular disorders showing the complex phenotype of peripheral neuropathy, myopathy, hearing loss, and hoarseness, which shows high inheritance and is caused by a single gene defect. Accordingly, recently developed therapeutic methods are applied at an early stage to maximize the therapeutic effects, and accurate diagnosis of the disease makes it possible to tailor therapy.

Hereinafter, the present invention will be described in more detail with reference to Examples. However, these Examples are for illustrative purposes only, and the invention is not intended to be limited by these Examples.

### Example 1

### Clinical Assessments

The study concluded leading to the present invention included a total of 33 members (15 affected patients and 18 unaffected patients) of a Korean autosomal dominant family with a complex phenotype of peripheral neuropathy, distal myopathy, hoarseness, and hearing loss (family ID: FC317, FIG. 1), and 283 healthy controls, who had no clinical features and family history of neuromuscular disorders and hearing loss. Total DNA was extracted from peripheral blood samples using the QIAamp Blood DNA mini kit (Qiagen, Hilden, Germany). Informed consent was obtained from all participants and from the parents of patients younger than 18 years of age according to the protocol approved by the institutional review board for Ewha Womans University, Mokdong Hospital.

Clinical information was obtained through history taking, physical examinations, clinical observations, and electrophysiological investigations. Clinical observations included detailed neurological exams, including assessments of muscle weakness, sensory impairment, hearing loss, hoarseness, and reflexes. Electrocardiogram (ECG), echocardiography, and blood chemistry including creatine kinase (CK), lactic acid, and pyruvic acid were done. The laryngeal study was performed in four patients (III-6, 11, 13, and IV-10) using a flexible fiberoptic laryngoscope. To determine hearing loss, pure-tone audiometry with air and bone conduction was performed in 17 individuals (11 affected patients and 6 unaffected patients) after otoscopic examination using an AC40 audiometry (Interacoustic, Denmark).

The results of clinical assessment of 15 affected patients in family FC317 according to the present invention are shown in the following Table 1.

TABLE 1

| | Patient | | | | | | | |
|---|---|---|---|---|---|---|---|---|
| | III-4 | III-6 | III-8 | III-11 | III-13 | III-16 | IV-3 | IV-5 |
| Sex/current age (yrs) | M/52 | M/52 | M/48 | F/45 | F/41 | M/33 | M/31 | F/18 |
| Age of onset (yrs)^{a} | 12 | 11 | 10 | 12 | 13 | 9 | 10 | 12 |
| Disease duration (yrs) | 40 | 41 | 38 | 33 | 28 | 24 | 21 | 6 |
| Muscle weakness | D > P | D > P | D > P | Distal | Distal | Distal | Distal | Distal |
| Distal muscle atrophy | Severe | Severe | Severe | Severe | Moderate | Moderate | Severe | Mild |
| | (I > U) | (I > U) | (I > U) | (I > U) | (I > U) | (I > U) | (I > U) | (I > U) |
| Sensory loss | − | − | ± | − | − | − | − | − |
| Hearing loss | NA | + | + | + | + | − | NA | NA |
| Hoarseness | + | + | + | + | + | − | + | − |
| Cardiac involvement | NA | − | − | − | − | − | NA | NA |
| Foot deformity | + | + | + | + | + | + | + | + |
| Knee jerk reflex | Areflex | Areflex | Areflex | Areflex | Areflex | Areflex | Areflex | Normal |
| Additional symptom | − | Seizure | Tremor | − | − | Tremor | Tremor | Arthritis |
| Creatine kinase (IU/L)^{b} | NA | WNL | 2.5 fold | WNL | WNL | 1.4 fold | NA | NA |
| Lactic acid (mg/dl)^{c} | NA | 13.0 | NA | 19.0 | 9.0 | 11.0 | NA | NA |
| Pyruvic acid (mg/dl)^{d} | NA | 0.5 | NA | 1.0 | 0.7 | 0.7 | NA | NA |
| MRI of lower limb^{e} | | | | | | | | |
| Leg | NA | 3.9 ± 0.3 | 3.6 ± 0.5 | 3.4 ± 0.8 | 3.5 ± 0.6 | 2.5 ± 0.9 | NA | NA |
| Thigh | NA | 1.4 ± 0.7 | 1.1 ± 0.6 | 0.4 ± 0.5 | 0.3 ± 0.4 | 0 | NA | NA |
| Muscle biopsy^{f} | NA | Grouping^{g} | NA | NA | NA | Grouping^{g} | NA | NA |

| | Patient | | | | | | |
|---|---|---|---|---|---|---|---|
| | IV-7 | IV-8 | IV-9 | IV-10 | IV-11 | IV-13 | IV-14 |
| Sex/current age (yrs) | M/15 | F/11 | F/18 | M/16 | F/15 | M/15 | F/11 |
| Age of onset (yrs)^{a} | 10 | 9 | 13 | 12 | 14 | 5 | 7 |
| Disease duration (yrs) | 5 | 2 | 5 | 4 | 1 | 10 | 4 |
| Muscle weakness | Distal | Distal | Distal | Distal | Distal | Distal | Distal |
| Distal muscle atrophy | Mild | Absent | Mild | Mild | Absent | Mild | Mild |
| | (I > U) | | (I > U) | (L = U) | | (I > U) | (L = U) |
| Sensory loss | − | − | − | − | − | − | − |
| Hearing loss | − | − | − | − | − | + | NA |
| Hoarseness | − | − | − | + | − | + | − |
| Cardiac involvement | − | − | − | − | − | − | − |
| Foot deformity | − | − | − | + | − | + | − |
| Knee jerk reflex | Normal | Normal | Normal | Hyporeflex | Normal | Hyporeflex | Normal |
| Additional symptom | − | − | − | Seizure | − | − | − |
| Creatine kinase (IU/L)^{b} | WNL | WNL | WNL | 1.3 fold | WNL | 2.1 fold | NA |
| Lactic acid (mg/dl)^{c} | NA | NA | 10.0 | 13.0 | 7.0 | 12.0 | NA |
| Pyruvic acid (mg/dl)^{d} | NA | NA | 0.7 | 1.1 | 0.6 | 0.7 | NA |
| MRI of lower limb^{e} | | | | | | | |
| Leg | 0.2 ± 0.4 | 0.1 ± 0.3 | 0.3 ± 0.5 | 0.2 ± 0.4 | 0.1 ± 0.3 | 0.8 ± 1.0 | 0.3 ± 0.5 |
| Thigh | 0 | 0 | 0 | 0 | 0 | 0 | 0 |
| Muscle biopsy^{f} | NA | NA | NA | NA | NA | NA | NA |

Abbreviation:
D = distal muscle;
L = lower limb;
NA = not available;
P = proximal muscle;
U = upper limb;
WNL = within normal limit;
+/− = positive or negative finding;
± = equivocal finding.
^{a}Age at onset of distal muscle weakness.
^{b}Creatine kinase values are listed in relations to the upper limits of normal.
^{c, d}Reference range: 4.5-14.4 mg/dl and 0.3-0.9 mg/dl, respectively.
^{e}Degree of fatty infiltration was graded on a five-point scale (mean ± S.D.).
^{f}Muscle biopsy was done at vastus lateralis (III-6) and at gastrocnemius muscles (III-16).
^{g}Grouping of muscle fiber types and frequent paracrystalline inclusions.

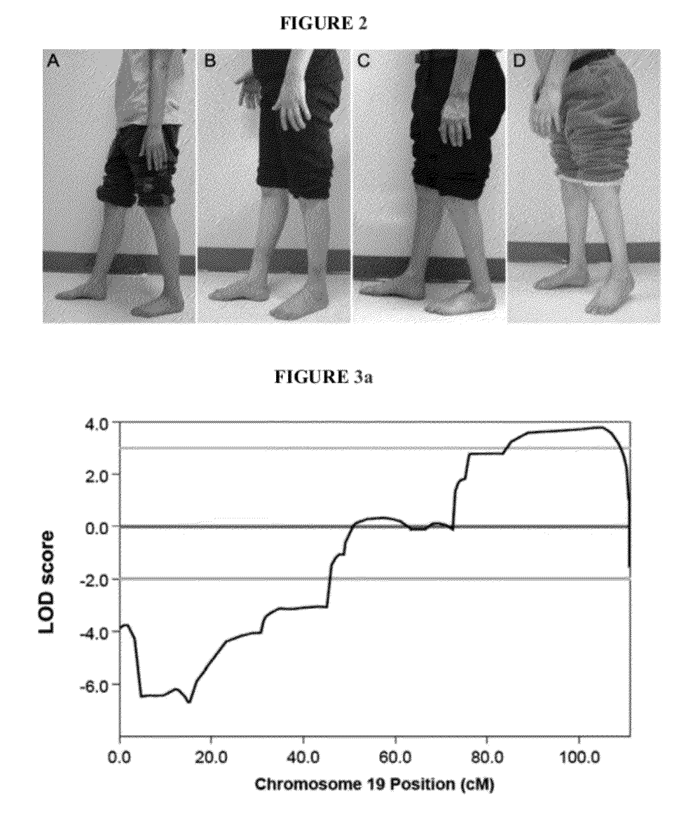

As shown in Table 1, muscle weakness and atrophy started and predominated in the distal portions of the legs, and were noted to a lesser extent distally in the upper limbs. Distal muscle weakness of the lower limbs varied from asymptomatic to severe, and the progressive leg muscle atrophy was associated with disease duration (FIG. 2). Mean age at onset of distal weakness was 10.6±2.4 years (range 5-14 years) and disease duration was 17.5±15.3 years (range 1-41 years). All 15 affected patients showed distal leg muscle weakness, and three patients (III-4, 6, and 8) showed mild proximal thigh muscle weakness. The frequency of foot deformities was high (67%), and the decreased knee jerk reflexes were found in the majority of the patients. Serum CK levels were normal to slightly elevated, which was consistent with the other autosomal dominant distal myopathies (Saperstein D S, et al., Muscle Nerve 24: 1440-1450, 2001). ECG or echocardiography was normal.

A hoarse voice was present in 8 of 15 affected individuals (53%), albeit some affected patients had a hypophonic voice only. Six patients (III-4, 6, 8, 11, 13, and IV-3) experienced hoarseness when they were in their twenties, and in individuals IV-10 and IV-13, a hoarse voice was observed at ages 15 and 10, respectively. However, difficulty with swallowing, aspiration, dyspnea, or ocular movement was not found, and flexible laryngoscope did not show paresis of the vocal cord.

The Nerve Conduction Velocities (NCV) of 12 affected patients are shown in the following Table 2.

TABLE 2

| | | Hearing loss in dB | | | | | | | |
|---|---|---|---|---|---|---|---|---|---|
| | | Right ear (kHz) | | | | Left ear (kHz) | | | |
| Patient | Sex/Age(yr) | 0.5 | 1 | 2 | 4 | 0.5 | 1 | 2 | 4 |
| III-6 | M/52 | 20 | 25 | 40 | 60 | 25 | 30 | 50 | 60 |
| III-8 | M/48 | 30 | 30 | 45 | 70 | 25 | 25 | 40 | 65 |
| III-11 | F/45 | 25 | 30 | 40 | 70 | 15 | 15 | 30 | 45 |
| III-13 | F/41 | 25 | 25 | 30 | 35 | 20 | 25 | 25 | 55 |
| III-16 | M/33 | 20 | 15 | 15 | 30 | 20 | 15 | 20 | 25 |
| IV-7 | M/15 | 15 | 20 | 15 | 10 | 15 | 15 | 15 | 10 |
| IV-8 | F/11 | 15 | 10 | 10 | 10 | 10 | 10 | 10 | 20 |
| IV-9 | F/18 | 15 | 20 | 15 | 10 | 15 | 20 | 20 | 10 |
| IV-10 | M/16 | 20 | 30 | 10 | 45 | 15 | 25 | 15 | 35 |
| IV-11 | F/15 | 5 | 5 | 5 | 5 | 5 | 5 | 0 | 5 |
| IV-13 | M/15 | 20 | 25 | 30 | 50 | 25 | 25 | 25 | 50 |

As shown in Table 2, audiological studies showed bilateral sensorineural hearing loss in 5 of 12 affected patients (45%). Hearing loss was present in individual III-6 from about age 25 and in individuals III-8, III-11, and III-13 when they were in their thirties. High-frequency loss was observed in individual IV-13 at age 12 during a general physical examination. Unaffected family members noticed no symptoms of hearing loss.

### Example 2

### Mapping of a New CMT Locus

<2-1> Genomewide SNP Linkage Scan

A genome-wide SNP linkage scan was performed on 28 members of family FC317 applying the Infinium II Human Linkage-12 Panel (Illumina, San Diego, Calif.). The chip included 6,090 SNP markers that are uniformly distributed on every chromosome with an average gap of 441 kbp and 0.58 cM. Genotyping data were scanned on the Illumina BeadStation 500G array scanner and analyzed with the Merlin-1.1.2 software using an autosomal dominant parametric model.

Genomewide SNP linkage analysis revealed a maximum multipoint LOD score of 3.79 at SNP marker rs1058511 under an autosomal dominant inheritance model (0.0001, 0.1, 0.9, 0.99). A haplotype cosegregated among affected patients that spans a region from approximately 75-110 Mbp (rs2041975 to rs1051500) on chromosome 19q13.2-3 (NCBI Build 36.1) (FIG. 3a). No additional chromosomal region showed LOD scores higher than 2 under an autosomal dominant model.

<2-2> Chromosomal Fine Mapping

Fine mapping of the chromosome 19q13 region was performed by genotyping 29 fluorescent-labeled microsatellites. PCR products were resolved on the automated genetic analyzer ABI3100, and data were analyzed using the Genotype program (Applied Biosystems, Foster City, Calif.). By applying the Mlink software, two-point LOD scores were obtained under an autosomal dominant model.

Fine mapping of the chromosomal linkage region revealed a two-point maximum LOD score of 6.360 under an autosomal dominant model (Theta: 0.00). Haplotype analysis narrowed the chromosomal region at 19q13.3 to approximately 13 cM (D19S412 to D19S601) (FIGS. 1 and 3b). The DMPK gene is close to this region, but could be excluded based on haplotype analysis (FIG. 3C). The linkage study also excluded main autosomal dominant CMT loci, such as 1p36.2 (MFN2; MIM #608507), 1q23.3 (MPZ; MIM #159440), 7p15 (GARS; MIM #600287), 7q11.23 (HSPB1; MIM #602195), 8p21.2 (NEFL; MIM #162280) 8q21.1 (GDAP1; MIM #606598), 9q34.13 (ALS4; MIM #602433), 10q21.2 (EGR2; MIM #129010), 12q24 (HSPB8; MIM #608014), 16p13.13 (LITAF; MIM #603795), 17p12 (PMP22; MIM #601097), and 19p13.2 (DNM2; MIM #602378).

### Example 3

### Identification of Missense Mutation in MYH14

<3-1> Mutation Screening

Sequencing analysis of all coding exons and flanking intronic sequences was performed on 34 candidate genes in the linkage region at 19q13.3 (FIG. 3c). In addition; mutational screening was performed on several previously reported genes with autosomal dominant CMT or distal myopathy. Particularly, tri-nucleotide extension was examined in the DMPK (MIM #605377) in DM1 locus and CNBP1 (ZNF9) (MIM #116955) in DM2 locus. The entire mitochondrial DNA (mtDNA) was also amplified and sequenced using the mitoSEQr resequencing system (Applied Biosystems). PCR products were sequenced on the automatic genetic analyzer ABI13100 using the BigDye terminator cycle sequencing kit (Applied Biosystems). In this regard, primer pairs represented by SEQ ID NOs: 3 to 92, as shown in the following Table 3, were designed to amplify all exons, and the promoter region of the MYH14 gene. Sequence variations were confirmed by analyzing both DNA strands. Nucleotides were counted by cDNA numbering with +1 corresponding to the A of the ATG initiation codon.

TABLE 3

| | | | SEQ | PCR |
|---|---|---|---|---|
| Target | | | ID | size |
| region | Name | sequence(5′->3′) | NO | (bp) |
| Promoter | P AF | AGAGTAGACTGTAGGGAGAGCAAGG | 3 | 499 |
| | P AR | AGAGGGTGTTAATTGCAGAAAGTC | 4 | |
| Promoter | P BF | CTTCCAACCTTGGGAAGTCTTT | 5 | 525 |
| | P BR | ATGAATGGGGGCCTTTGTAAG | 6 | |
| Promoter | P CF | GTACATGGTCTACGTTCGACAAAAG | 7 | 423 |
| | P CR | AGAGAGCAGCAGAGGCCAAT | 8 | |
| Exon 1 | N1F | ATTGGCCTCTGCTGCTCTCT | 9 | 239 |
| | N1R | AAGGTGAGTGTCCGCGTCA | 10 | |
| Exon 2 | 2F | GAATGAAATGAGTAAGCTGGGTCT | 11 | 684 |
| | 2R | GGATACAGATGATAAACAGCCTCAA | 12 | |
| Exon 3 | 3F | GTTATGGTGTAGACATACCATGTGC | 13 | 391 |
| | 3R | AAGTCTACAAGCTGTCATTTGACCT | 14 | |
| Exon 4 & | 4-5F | GTTACACATCAAGACCCAAGCTTTT | 15 | 499 |
| 5 | | | | |
| | 4-5R | ACCTCCATGAGGGGAAGAGA | 16 | |
| Exon 6 | 6F | ATCACTGGTTCACCTGTGTGTCT | 17 | 187 |
| | 6R | CCTGTCTACCAAGAAGATCATGC | 18 | |
| Exon 7 | 7F | ATACTCAGTCAGCTGGAGACACAG | 19 | 296 |
| | 7R | CCCCTCCTCCCTCAACAG | 20 | |
| Exon 8 | 8F | GGGTTTGGGCTGTTGTTCAC | 21 | 222 |
| | 8R | AGTGGGAGGTGCTTTCCATAC | 22 | |
| Exon 9 | 9F | ACTCCACTACACCACAGGAGAGA | 23 | 300 |
| | 9R | TCTTGCTTCCTCCCCAGAAG | 24 | |
| Exon 10 | 10F | GATGAATCCAGGATGAGTCTGA | 25 | 292 |
| | 10R | AAAGAGATCGGGGCATGAAT | 26 | |
| Exon 11 | 11F | AGGGGTGGTGATATAATTTGCCTTA | 27 | 299 |
| | 11R | ATGCTCTCTACGTGGGACAGG | 28 | |
| Exon 12 | 12F | AAACGGTACCCTCTCCCTTG | 29 | 284 |
| | 12R | GGTGGGAAAAACAGCATACACT | 30 | |
| Exon 13 | 13F | GCATTGTTCTTGATGGTACTTACAC | 31 | 348 |
| | 13R | AGGACCGGAGTGAGGAAGTT | 32 | |
| Exon 14 | N14F | CTCTCCCCTTCTCCCTGGTC | 33 | 845 |
| | N14R | GTGAGAGCTCTGATTAACCGATTG | 34 | |
| Exon 15 | 15F | TAGAGTCGGGGGTTTCACTGT | 35 | 474 |
| | 15R | CCATCTGTAGCCAGTGGAGAT | 36 | |
| Exon 16 | 16F | GAAGGCTCCTTAGGAAATCCAG | 37 | 299 |
| | 16R | CAGAAAACTCAGGTTCAGACCTC | 38 | |
| Exon 17 | 17F | GATATCTACCTTACAGGCTGTGTGA | 39 | 476 |
| | 17R | CTGAGCCTAGGAATAGAAGCAATTT | 40 | |
| Exon 18 | 18F | GAAACAGGAAATTGCCAAGC | 41 | 299 |
| | 18R | AGAGTGGGTGGAGACCTGAAT | 42 | |
| Exon 19 | 19F | CTTGTTATTGTCACTGTTGTTCCTG | 43 | 454 |
| | 19R | AGGTAATGAGGAAGGTCAATGAAG | 44 | |
| Exon 20 | N20F3 | CCCATTACTCCCCCTCCTCACC | 45 | 318 |
| | 20R | ACACCTAGAGCCATCTGGTCAAC | 46 | |
| Exon 21 | 21F | AGCTTGGCTCTCTTGCTAAGG | 47 | 230 |
| | 21R | AAGGCCCTATCCTCTCCCTACT | 48 | |
| Exon 22 | 22F | CAGCTAATAGGTGGAGGAGAAGG | 49 | 400 |
| | 22R | GCCTCAGTACACCTTAGCTTTGC | 50 | |
| Exon 23 | 23F | GAACTTAAAGGCCAAAGCAAGTTAC | 51 | 390 |
| | 23R | ACACACGGTTTGTAGAAGCAAAG | 52 | |
| Exon 24 | 24F | TTCCCCATCACACTCCATCT | 53 | 300 |
| | 24R | GAGAACCGCAGTGTGGTAAC | 54 | |
| Exon 25 | 25F | AGATGAGAACAACACCAGAAGC | 55 | 300 |
| | 25R | ACTGAGGCCCTGTAGCACAT | 56 | |
| Exon 26 | 26F | TGAGCTTAGCCTTATATGTGACTGG | 57 | 397 |
| | 26R | CTGTCTGTGGCTGGTGAGTG | 58 | |
| Exon 27 | 27F | AGAGAGAGGATTCTAACCTGGGACT | 59 | 470 |
| | 27R | ACCTAGAGCCGGTGGTTCAT | 60 | |
| Exon 28 | 28F | GAGGCCCTCATATTTTAAGGAAAC | 61 | 360 |
| | 28R | GGAGATGAGCTAGCCTAGAACCAG | 62 | |
| Exon 29 | N29F | GAGTGAGGGGTGAGCTATTTGTT | 63 | 494 |
| | N29R | CTGTGCCCAGACTTGTACATGATTA | 64 | |
| Exon 30 & | 30- | GTGTGGTAGAGAGTTGAGGCAGA | 65 | 573 |
| 31 | 31F | | | |
| | 30- | GTAAGGGGGAGTCAGAGATGAGA | 66 | |
| | 31R | | | |
| Exon 32 | 32F | CAGCTTACACCTTGGTCACTCAT | 67 | 392 |
| | 32R | GATTACTGGTTCCTCACAACGAC | 68 | |
| Exon 33 | 33F | CTTGTGTCTCTGCCTTTGTCTG | 69 | 398 |
| | 33R | CATGTACGTGTCTCCCCTCAC | 70 | |
| Exon 34 | N34F | ACTAGGATGGGCACACTGACTT | 71 | 400 |
| | N34R | CCTGTACACACCATGGCATAC | 72 | |
| Exon 35 | 35F | GACCAAGTAAAGAGAGTCAGGGAGT | 73 | 388 |
| | 35R | AGCTAGCCTTAGACCCTGGAGT | 74 | |
| Exon 36 | 36F | GAGAGTTCCCTGCTTGTTCAC | 75 | 490 |
| | 36R | ACATGGTAAGACACACCCACCT | 76 | |
| Exon 37 | 37F | TTACCCAGGGACAGCATGAG | 77 | 400 |
| | 37R | CCTTGCCTCACACTACAGGAC | 78 | |
| Exon 38 | 38F | ACAGGGTCCTGTAGTGTGAGG | 79 | 327 |
| | 38R | CTATTCAACCTTCAAAACCCAACTC | 80 | |
| Exon 39 | 39F | CAGTGCACTTAATTCTCAGAGGTG | 81 | 391 |
| | 39R | CTAAACCACGTGTTTGTAACACAGC | 82 | |
| Exon 40 | 40F | ATGAGTCTATGGGGACAAAATCCTA | 83 | 241 |
| | 40R | CTGTGCAATTTCTCAGCCAGT | 84 | |
| Exon 41 | 41F | GATGGGGCAGAGAGAGTCAG | 85 | 393 |
| | 41R | ATCCCATTTCCCCCTGTTGT | 86 | |
| Exon 42 | 42F | CCCTTATTTTGTTCTCTCTCTTCC | 87 | 390 |
| | 42R | ATTTCCAAAGGGCAAGAAGTC | 88 | |
| 3′-UTR | 3UTR | CCAGGTCTTCCGACTAGAGGAG | 89 | 592 |
| | AF | | | |
| | 3UTR | TCCTAAAAAGATGCACAGAGAGAC | 90 | |
| | AR | | | |
| 3′-UTR | 3UTR | CATTCCCTCTGCTTCTCTCTC | 91 | 500 |
| | BF | | | |
| | 3UTR | CAGGTGTCATTCTAACCAGCAG | 92 | |
| | BR | | | |

Because CMT4F (PRX; MIM #605725) and CMT2B2 (MED25; MIM #610197) loci are located near or within the disequilibrium region (Berghoff C, et al., Neuromuscul Disord 5: 301-306, 2004; Kabzinska D, et al., Neurology 66: 745-747, 2006; Leal A, et al., Neurogenetics 10: 275-287, 2009), the present inventors carefully examined both genes, but did not identify significant nucleotide changes. Similarly, no causative mutation was found in EMP3 (MIM #602335), which is located within the linkage region and shows highly conserved homology with PMP22 myelin gene (CMT1A). Although the DMPK gene at the DM1 locus was excluded by the haplotype analysis, the careful examination of DMPK revealed neither abnormal CTG repeats nor a causative mutation in the coding regions.

<3-2> Determination of Copy Number Variation

Copy number variations (CNV) in the chromosome 19q13 region were determined by using a custom-designed high-density comparative genomic hybridization (CGH) 135K microarray (Roche-NimbleGen, Madison, Wis.). The array covered a region on chromosome 19 between 36606560-60238000 bp (UCSC hg18, NCBI Build 36.1). The mean probe size and spacing length of the array were 60-mer and 215 bp, respectively. The CGH data were analyzed with NimbleScan (ver. 2.4) and SignalMap (ver. 1.9) softwares. The gain and loss threshold used in this study were log₂ ratio>0.3 and <−0.3, respectively.

The present inventors also excluded chromosomal duplication and deletion events in the 19q13 region by applying a custom-designed high-density CGH array to three individuals of the family FC317 (two affected patients and one unaffected patient) (FIG. 4a).

<3-3> Karyotyping and MYH14 Expression in Muscle

Karyotyping was performed using cultured metaphase white blood cells obtained from proband (III-13) and her sibling (III-16). Metaphase chromosomes were visualized with Gimsa staining. MYH14 expression in biopsied gastrocnemius muscle from a patient (III-16) was determined by quantitative real-time PCR using the QuantiTect Primer Assay (QT00080248, Qiagen).

The karyotyping analysis in each affected male and female individual revealed no chromosomal abnormality (FIG. 4a).

Taken together, in the screen of 34 candidate genes, the only functionally significant variant identified was a missense change in MYH14, which encodes the non muscle myosin heavy chain 14 (MYH14) protein. The mutation was a guanine (G) to thymine (T) transversion at position 2281 in exon 23 of the MYH14 gene (NM_{—}001077186.1) (c.2282G>T), which leads to substitution of an arginine residue at position 941 with leucine (p.Arg941Leu) (FIG. 3d). The c.2282G>T mutation in the MYH14 gene has not been previously reported in the dbSNP database (ncbi.nlm.nih.gov/snp/). This mutation completely co-segregated with all affected members in the pedigree. Sequencing analysis confirmed the absence of this variant in unaffected members of the pedigree and in 566 healthy control chromosomes. The mutation is located in the tail domain of MYH14 and it is highly conserved in other species (FIG. 3e).

Although MYH14 expression in skeletal muscle, cochlea, brain, and peripheral nerves has been reported (Leal A, et al., Gene 312: 165-171, 2003; Donaudy F, et al., Am J Hum Genet 74: 770-776, 2004; Golomb E, et al., J Biol Chem 279: 2800-2808, 2004), the present inventors confirmed MYH14 expression in biopsied gastrocnemius muscle by quantitative real-time PCR. The expression level in gastrocnemius muscle from an affected member (III-16) was slightly decreased compared to a normal male (FIG. 5).

### Example 4

### Electrophysiological Studies

Electrophysiological studies were carried out on 12 affected individuals (6 males and 6 females). Motor NCVs of the median, ulnar, peroneal, and tibial nerves were determined. Amplitudes of compound muscle action potential (CMAP) were measured from positive to negative peak values. Sensory NCVs were obtained from the median, ulnar, and sural nerves by an orthodromic scoring. Amplitudes of sensory nerve action potential (SNAP) were measured from positive peaks to negative peaks. An electromyography (EMG) was performed in the first dorsal interosseous, biceps brachii, tibialis anterior, medial gastrocnemius, and vastus lateralis muscles.

The electrophysiological findings of 29 nerves in 12 affected patients (6 males and 6 females) are shown in the following Table 4.

TABLE 4

| | | | Median | | Ulnar | | Peroneal | | Tibial | | Median | | Ulnar | | Sural | |
|---|---|---|---|---|---|---|---|---|---|---|---|---|---|---|---|---|
| | Age | | motor | | motor | | motor | | motor | | sensory | | sensory | | sensory | |
| Patient | (yrs) | R/L | Amp | CV | Amp | CV | Amp | CV | Amp | CV | Amp | CV | Amp | CV | Amp | CV |
| III-6 | 50 | R | 4.2 | 56.4 | 7.9 | 58.3 | A | A | 0.6 | 41.1 | 37.3 | 42.3 | 22.6 | 38.7 | 31.4 | 38.5 |
| | 50 | L | 6.3 | 57.4 | 6.4 | 70.0 | A | A | 0.6 | 49.3 | 24.2 | 44.1 | 22.3 | 39.1 | 20.4 | 38.7 |
| | 52 | R | 4.8 | 56.5 | 5.9 | 56.4 | A | A | 0.7 | 45.6 | 33.5 | 44.1 | 24.9 | 40.4 | 22.2 | 39.3 |
| | 52 | L | 6.0 | 52.8 | 6.6 | 54.3 | A | A | 0.5 | 44.3 | 36.7 | 45.5 | 24.6 | 39.2 | 30.8 | 39.5 |
| III-8 | 48 | R | 2.9 | 53.5 | 6.1 | 54.1 | A | A | 12.5 | 36.1 | 39.8 | 35.7 | 31.7 | 34.2 | 23.4 | 30.3 |
| | 48 | L | 5.4 | 59.8 | 5.1 | 55.3 | A | A | 7.7 | 37.5 | 32.2 | 38.3 | 18.6 | 34.5 | 28.2 | 28.1 |
| III-11 | 45 | L | 7.9 | 58.8 | 5.2 | 69.0 | 1.5 | 49.3 | 7.3 | 47.7 | 41.0 | 46.9 | 32.0 | 41.7 | 30.3 | 42.3 |
| III-13 | 40 | R | 10.3 | 56.1 | 9.9 | 56.4 | A | A | 16.5 | 45.6 | 47.8 | 42.3 | 23.6 | 41.7 | 24.4 | 39.5 |
| | 40 | L | 6.9 | 59.5 | 7.7 | 63.9 | A | A | 14.7 | 43.8 | 59.1 | 42.9 | 28.7 | 39.1 | 24.0 | 34.1 |
| | 41 | R | 11.0 | 53.5 | 11.0 | 55.7 | A | A | 17.0 | 46.8 | 57.1 | 41.1 | 26.2 | 40.3 | 24.1 | 35.7 |
| | 41 | L | 7.3 | 53.7 | 7.3 | 57.5 | A | A | 16.8 | 43.3 | 49.0 | 41.7 | 22.6 | 39.1 | 22.3 | 36.1 |
| III-16 | 33 | R | 7.1 | 59.0 | 7.0 | 64.7 | A | A | 10.6 | 48.1 | 43.7 | 48.4 | 26.0 | 40.3 | 27.1 | 41.2 |
| | 33 | L | 8.2 | 57.3 | 4.8 | 64.7 | A | A | 7.5 | 48.6 | 57.8 | 46.9 | 33.9 | 43.8 | 29.4 | 42.2 |
| IV-7 | 14 | R | 6.2 | 53.4 | 18.1 | 54.8 | 4.6 | 43.9 | 15.1 | 42.2 | 22.3 | 40.1 | 14.6 | 43.9 | 10.2 | 33.1 |
| | 14 | L | 7.3 | 54.5 | 16.2 | 60.5 | 5.9 | 37.7 | 12.1 | 38.6 | 22.0 | 43.4 | 15.9 | 37.7 | 12.9 | 34.8 |
| | 15 | R | 7.0 | 52.2 | 17.8 | 52.4 | 3.7 | 41.5 | 13.2 | 44.7 | 26.1 | 41.7 | 8.4 | 37.9 | 11.4 | 35.3 |
| | 15 | L | 8.4 | 55.3 | 17.8 | 53.7 | 5.1 | 37.5 | 14.1 | 38.4 | 19.2 | 40.5 | 13.4 | 38.6 | 11.9 | 38.2 |
| IV-8 | 11 | R | 6.5 | 57.2 | 6.3 | 54.7 | 0.3 | 44.2 | 14.7 | 49.3 | 31.8 | 41.7 | 34.6 | 38.8 | 27.2 | 40.5 |
| | 11 | L | 8.1 | 56.1 | 4.5 | 58.1 | 0.2 | 46.9 | 10.8 | 48.0 | 45.1 | 41.7 | 32.4 | 41.7 | 28.6 | 41.7 |
| IV-9 | 18 | R | 15.4 | 63.2 | 10.0 | 60.8 | 3.4 | 47.8 | 8.2 | 47.3 | 55.0 | 40.4 | 35.3 | 39.5 | 29.4 | 36.6 |
| | 18 | L | 11.5 | 58.3 | 14.0 | 63.6 | 4.6 | 45.1 | 8.0 | 47.9 | 53.7 | 43.0 | 26.1 | 40.3 | 31.6 | 33.4 |
| IV-10 | 16 | R | 12.3 | 60.8 | 11.6 | 66.2 | 0.2 | 47.6 | 7.0 | 48.6 | 27.0 | 42.3 | 15.7 | 43.1 | 27.6 | 45.5 |
| | 16 | L | 14.7 | 64.7 | 10.6 | 67.5 | 0.6 | 48.6 | 7.1 | 47.2 | 41.2 | 43.5 | 32.4 | 41.7 | 30.5 | 47.8 |
| IV-11 | 15 | R | 10.6 | 56.3 | 14.5 | 62.1 | 2.9 | 49.2 | 14.6 | 47.8 | 30.9 | 40.4 | 19.7 | 39.5 | 18.1 | 35.5 |
| | 15 | L | 10.5 | 64.3 | 13.9 | 62.9 | 3.3 | 45.7 | 12.7 | 45.7 | 30.0 | 41.7 | 20.7 | 39.5 | 21.5 | 37.2 |
| IV-13 | 15 | R | 7.8 | 53.0 | 5.4 | 53.7 | A | A | 14.7 | 45.1 | 31.9 | 37.8 | 18.0 | 33.1 | 24.1 | 38.5 |
| | 15 | L | 5.2 | 55.3 | 4.1 | 57.9 | A | A | 10.5 | 46.2 | 35.0 | 36.9 | 17.9 | 35.7 | 26.7 | 38.2 |
| IV-14 | 11 | R | 9.1 | 52.9 | 11.4 | 56.1 | 2.1 | 42.9 | 12.5 | 43.8 | 31.5 | 40.4 | 22.4 | 39.4 | 14.3 | 36.0 |
| | 11 | L | 10.6 | 56.1 | 6.9 | 57.3 | 1.6 | 42.8 | 15.0 | 43.4 | 31.5 | 41.7 | 20.4 | 40.3 | 17.2 | 36.8 |

Boldface represents abnormal values.
A = absent response;
Age = age at examination;
Amp = amplitude (motor: by mV; sensory: conduction velocity (m/sec);
R/L = right/left.
Normal CVs: motor median ≧50.5, ulnar ≧51.1, peroneal ≧41.2, tibial ≧41.1, sensory median ≧39.3, ulnar ≧37.5, and sural ≧32.1.
Normal amplitudes: motor median ≧6; ulnar ≧8, peroneal ≧6, tibial ≧6, sensory median ≧8, ulnar ≧7.9, and sural ≧6.0.

As shown in Table 4, nerve conduction studies demonstrated mildly reduced or normal median, ulnar, and sural sensory NCVs, and reduced median and ulnar CMAPs were always associated with normal NCVs. Noteworthy, severely reduced CMAPs were observed in bilateral peroneal nerves.

In addition, EMG was performed in 12 patients, and they showed fibrillation potentials, but myotonic discharges were not found. Two affected patients (IV-8 and -11) did not show distal muscle atrophy and fibrillation potentials in the tibialis anterior muscles. In the same affected patients, the present inventors observed both a large amplitude with long duration motor unit action potential (MUAP), which was usually seen in neuropathy (FIG. 6A), and also a small amplitude with short duration polyphasic MUAP seen in myopathy (FIG. 6B). These findings suggested that the affected patients have evidence of both neuropathy and myopathy.

### Example 5

### Sequential Fatty Infiltration in Distal Muscle

Fourteen individuals (12 affected patients and 2 unaffected patients) were studied with an MRI of the lower limbs using a 1.5-T system (Siemens Vision, Siemens, Germany). Lower leg imaging was carried out in axial and coronal planes applying the following protocols: T1-weighted spin-echo'(SE) (TR/TE 570-650/14-20, 512 matrixes), T2-weighted SE (TR/TE 2800-4000/96-99, 512 matrixes), and fat-suppressed T2-weighted SE (TR/TE 3090-4900/85-99, 512 matrixes). Muscles were graded on a five-point scale, as follows: 0=no fat signal in muscle, 1=some fatty streaks, 2=fat occupying a minor part of muscle, 3=similar amount of fat and muscle tissue, and 4=fat occupying the greater part of muscle.

All examined affected individuals showed abnormal fatty infiltrations on magnetic resonance imaging (MRI) (Table 1). Eight of 12 patients showed fatty replacements of muscle tissue only in their legs, and all of them had anterior compartment involvement. Four patients (III-6, 8, 11, and 13) showed both leg and thigh muscle involvement; in all these patients, the posterior compartment of the thighs was involved, and in two, the anterior and medial compartments were involved. In addition, a sequential pattern of onset of muscle involvement associated with disease duration was observed. In the early stage of the disease, fatty infiltrations were present in the anterior and lateral compartments of the legs (FIGS. 7A-B), and in later stages, posterior compartment leg muscles were also affected, and muscle atrophy was noticed (FIGS. 7C-D).

### Example 6

### Histopathologic Findings

Muscle biopsies of left vastus lateralis and right lateral gastrocnemius were performed in patients III-6 and III-16, respectively. Serial frozen sections were stained with hematoxylin and eosin (H-E), NADH-tetrazolium reductase (NADH-TR), succinate dehydrogenase (SDH), modified Gomori trichrome (mGT), periodic acid-Schiff (PAS), Oil-red-O, and ATPase reaction with different pH preincubation. Immunostaining of myosin heavy chain (fast) (NCL-MHCf, monoclonal, 1:20) and myosin heavy chain (slow) (NCL-MHCs, monoclonal, 1:40: Vision Biosystems, Newcastle, UK), dystrophin, sarcoglycan, dysferlin, and titin was done. For electron microscopic observation, specimens were fixed in 2% glutaraldehyde in 25 mM cacodylate buffer (pH 7.4), and processed for semithin and ultrathin studies.

The histopathologic features of the muscle biopsies were similar in two patients (III-6 and III-16). Myofibers showed moderate to marked variation of fiber size and shape (FIG. 8A). Immunostaining with dystrophin, sarcoglycan, dysferin, and titin did not show abnormal findings, and no inflammatory infiltration was present. The grouping of the histochemical muscle fiber types were observed by ATPase with pH 9.4 preincubation and immunostaining with myosin heavy chain (fast), myosin heavy chain (slow), and myosin IIa (FIG. 8B). NADHTR and SDH staining showed multifocal subsarcolemmal accumulation of mitochondria, although mGT staining did not show ragged red fibers or rimmed vacuoles. Notably, the electron micrographs frequently revealed subsarcolemmal accumulation of enlarged mitochondria with variable sized rectangular or elongated rhomboidal paracrystalline inclusions (FIG. 8C).

What is claimed is:

1. A mutated MYH14 gene having a substitution of guanine by thymine at nucleotide position 2822 of SEQ ID NO: 1.

2. A mutated Myh14 protein encoded by the mutated MYH14 gene according to claim 1.

3. The mutated Myh14 protein according to claim 2, wherein the protein has a substitution of an arginine residue with guanine at amino acid position 941 of SEQ ID NO: 2.

4. A diagnostic composition for inherited neuromuscular disorders showing a complex phenotype of peripheral neuropathy, myopathy, hearing loss, and hoarseness, comprising an agent capable of detecting the expression of mRNA of the mutated MYH14 gene according to claim 1 or a protein encoded by the gene in a sample of an individual.

5. The diagnostic composition according to claim 4, wherein the expression of mRNA of the mutated MYH14 gene or a protein encoded by the gene is specifically detected in a sample of an individual with a complex phenotype of peripheral neuropathy, myopathy, hearing loss, and hoarseness.

6. The diagnostic composition according to claim 4, wherein the agent capable of detecting the mRNA expression is a pair of primers or probes specifically binding to the mutated MYH14 gene.

7. The diagnostic composition according to claim 6, wherein a pair of primers specifically binding to the mutated MYH14 gene has the base sequences represented by SEQ ID NOs: 51 and 52.

8. The diagnostic composition according to claim 4, wherein the agent capable of detecting the protein expression is an antibody specific to the protein encoded by the mutated MYH14 gene.

9. A diagnostic kit for inherited neuromuscular disorders showing a complex phenotype of peripheral neuropathy, myopathy, hearing loss, and hoarseness, comprising the composition of claim 4.

10. The diagnostic kit according to claim 9, wherein the kit is an RT-PCR kit, a microarray chip kit, or a protein chip kit.

11. The diagnostic kit according to claim 10, wherein the RT-PCR kit includes a pair of primers specifically binding to the mutated MYH14 gene.

12. The diagnostic kit according to claim 11, wherein a pair of primers specifically binding to the mutated MYH14 gene has the base sequences represented by SEQ ID NOs: 51 and 52.

13. The diagnostic kit according to claim 9, wherein the microarray chip kit includes probes specifically binding to the mutated MYH14 gene.

14. The diagnostic kit according to claim 9, wherein the protein chip kit includes an antibody specific to the protein encoded by the mutated MYH14 gene.

15. A method for diagnosing inherited neuromuscular disorders showing a complex phenotype of peripheral neuropathy, myopathy, hearing loss, and hoarseness, comprising the following steps of:
1) measuring mRNA expression of the mutated MYH14 gene or expression of the protein encoded by the gene in a sample of an individual; and
2) determining that the individual has a high risk of inherited neuromuscular disorders showing the complex phenotype of peripheral neuropathy, myopathy, hearing loss, and hoarseness, when the mRNA expression of the mutated MYH14 gene or expression of the protein encoded by the gene is detected in the sample.

16. The method according to claim 15, wherein the mRNA expression of step 1) is measured by using a pair of primers or probes specifically binding to the mutated MYH14 gene.

17. The method according to claim 16, wherein a pair of primers specifically binding to the mutated MYH14 gene has the base sequences represented by SEQ ID NOs: 51 and 52.

18. The method according to claim 15, wherein the mRNA expression of step 1) is measured by an analysis method selected from the group consisting of reverse transcription polymerase chain reaction, competitive reverse transcription polymerase chain reaction, real-time reverse transcription polymerase chain reaction, RNase protection assay (RPA), Northern blotting, and DNA chip assay.

19. The method according to claim 15, wherein the protein expression of step 1) is measured by using an antibody specific to the protein encoded by the mutated MYH14 gene.

20. The method according to claim 15, wherein the protein expression of step 1) is measured by an analysis method selected from the group consisting of Western blotting, ELISA, radioimmunoassay, radialimmunodiffusion, Ouchterlony immunodiffusion, rocket immunoelectrophoresis, immunohistostaining, immunoprecipitation assay, complement fixation assay, FACS, and protein chip assay.


SEQUENCE LISTING (92 sequences)

SEQ ID NO: 1 - DNA, 6831 bp, Homo sapiens
  gene (1)..(6831): MYH14
ctctttctccccaggccgaagcctcgggacggccctggaagccgaccatggcagccgtgaccatgtcggtgcccgggcggaaggcgccccccaggccgggcccagtgcccgaggcggcccagccgttcctgttcacgccccgcgggcccagcgcgggtggcgggcctggctcgggcacctccccgcaggtggagtggacggcccggcgtctcgtgtgggtgccttcggagcttcacgggttcgaggcggcggcgctgcgggacgaaggcgaggaggaggcggaggtggagctggcggagagcgggaggcggctgcgactgccgcgggaccagatccagcgcatgaacccgcccaagttcagcaaggccgaggacatggccgagctgacctgcctcaacgaggcctcggtcctgcacaacctccgggagcggtactactccggcctcatctacacgtactccggccttttctgtgtggtcatcaacccgtacaagcagcttcccatctacacagaagccattgtggagatgtaccggggcaagaagcgccacgaggtgccaccccacgtgtacgcagtgaccgagggggcctatcggagcatgctgcaggatcgtgaggaccagtccattctctgcactggagagtctggagctgggaagacggaaaacaccaagaaggtcatccagtacctcgcccacgtggcgtcgtctccaaagggcaggaaggagccgggtgtccccgcctccgtcagcaccgtgtcttatggtgagctggagcggcagctgcttcaggccaaccccatcctagaggcctttggcaatgccaagacagtgaagaatgacaactcctcccgattcggcaaattcatccgcatcaactttgatgttgccgggtacatcgtgggcgccaacattgagacctacctgctggagaagtcgcgggccatccgccaggccaaggacgagtgcagcttccacatcttctaccagctgctggggggcgctggagagcagctcaaagccgacctcctcctcgagccctgctcccactaccggttcctgaccaacgggccgtcatcctctcccggccaggagcgggaactcttccaggagacgctggagtcgctgcgggtcctgggattcagccacgaggaaatcatctccatgctgcggatggtctcagcagttctccagtttggcaacattgccttgaagagagaacggaacaccgatcaagccaccatgcctgacaacacagctgcacagaagctctgccgcctcttgggactgggggtgacggatttctcccgagccttgctcacccctcgcatcaaagttggccgagactatgtgcagaaagcccagactaaggaacaggctgacttcgcgctggaggccctggccaaggccacctacgagcgcctcttccgctggctggttctgcgcctcaaccgggccttggaccgcagcccccgccaaggcgcctccttcctgggcatcctggacatcgcgggctttgagatcttccagctgaactccttcgagcagctctgcatcaactacaccaacgagaagctgcagcagctcttcaaccacaccatgttcgtgctggagcaggaggagtaccagcgtgagggcatcccctggaccttcctcgactttggcctcgacctgcagccctgcatcgacctcatcgagcggccggccaacccccctggactcctggccctgctggatgaggagtgctggttcccgaaggccacagacaagtcgtttgtggagaaggtagcccaggagcagggcggccaccccaagttccagcggccgaggcacctgcgggatcaggccgacttcagtgttctccactacgcgggcaaggtcgactacaaggccaacgagtggctgatgaaaaacatggaccctctgaatgacaacgtcgcagccttgctccaccagagcacagaccggctgacggcagagatctggaaagacgtggagggcatcgtggggctggaacaggtgagcagcctgggcgacggcccaccaggtggccgcccccgtcggggtatgttccggacagtgggacagctctacaaggagtccctgagccgcctcatggccacactcagcaacaccaaccccagttttgtccgctgcattgtccccaaccacgagaagagggccgggaagctggagccacggctggtgctggaccagcttcgctgcaacggggtcctggagggcatccgcatctgtcgccagggcttccccaaccgcatcctcttccaggagttccggcagcgatacgagatcctgacacccaatgccatccccaagggcttcatggatgggaagcaggcctgtgaaaagatgatccaggcgctggaactggaccccaacctctaccgcgtgggacagagcaagatcttcttccgggctggggtcctggcccagctggaagaggagcgagacctgaaggtcaccgacatcatcgtctccttccaggcagctgcccggggatacctggctcgcagggccttccagaagcgccagcagcagcagagcgccctgagggtgatgcagcggaactgcgcggcctacctcaagctgagacactggcagtggtggcggctgtttaccaaggtgaagccactgctgcaggtgacgcggcaggatgaggtgctgcaggcacgggcccaggagctgcagaaagtgcaggagctacagcagcagagcgcccgcgaagttggggagctccagggccgagtggcacagctggaagaggagcgcgcccgcctggcagagcaattgcgagcagaggcagaactgtgtgcagaggccgaggagacgcgggggaggctggcagcccgcaagcaggagctggagctggtggtgtcagagctggaggctcgcgtgggcgaggaggaggagtgcagccgtcaaatgcaaaccgagaagaagaggctgcagcagcacatacaggagctagaggcccaccttgaggctgaggagggtgcgcggcagaagctgcagctggagaaggtgacgacagaggcaaaaatgaagaaatttgaagaggacctgctgctcctggaagaccagaattccaagctgagcaaggagcggaagctgctggaagatcgtctggccgagttctcatcccaggcagctgaggaggaggagaaggtcaagagcctcaataagctacggctcaaatatgaggccacaatcgcagacatggaggaccgcctacggaaggaggagaagggtcgccaggagctggagaagctgaagcggaggctggatggggagagctcagagctgcaggagcagatggtggagcagcaacagcgggcagaggagctgcgggcccagctgggccggaaggaggaggagctgcaggctgccctggccagggcagaagacgagggtggggcccgggcccagctgctgaaatccctgcgggaggctcaagcagccctggccgaggcccaggaggacctggagtctgagcgtgtggccaggaccaaggcggagaagcagcgccgggacctgggcgaggagctggaggcgctgcggggcgagctggaggacacgctggactccaccaacgcacagcaggagctccggtccaagagggaacaggaggtgacggagctgaagaagactctggaggaggagactcgcatccacgaggcggcagtgcaggagctgaggcagcgccacggccaggccctgggggagctggcggagcagctggagcaggcccggaggggcaaaggtgcatgggagaagacccggctggccctggaggccgaggtgtccgagctgcgggcagaactgagcagcctgcagactgcacgtcaggagggtgagcagcggaggcgccgcctggagttacagctgcaggaggtgcagggccgggctggtgatggggagagggcacgagcggaggctgctgagaagctgcagcgagcccaggctgaactggagaatgtgtctggggcgctgaacgaggctgagtccaaaaccatccgtcttagcaaggagctgagcagcacagaagcccagctgcacgatgcccaggagctgctgcaggaggagaccagggcgaaattggccttggggtcccgggtgcgagccatggaggctgaggcagccgggctgcgtgagcagctggaggaggaggcagctgccagggaacgggcgggccgtgaactgcagactgcccaggcccagctttccgagtggcggcggcgccaggaggaggaggcaggggcactggaggcaggggaggaggcacggcgccgggcagcccgggaggccgaggccctgacccagcgcctggcagaaaagacagagaccgtggatcggctggagcggggccgccgccggctgcagcaggagctggacgacgccaccatggacctggagcagcagcggcagcttgtgagcaccctggagaagaagcagcgcaagtttgaccagcttctggcagaggagaaggcagctgtacttcgggcagtggaggaacgtgagcgggccgaggcagagggccgggagcgtgaggctcgggccctgtcactgacacgggcactggaggaggagcaggaggcacgtgaggagctggagcggcagaaccgggccctgcgggctgagctggaggcactgctgagcagcaaggatgacgtcggcaagagcgtgcatgagctggaacgagcctgccgggtagcagaacaggcagccaatgatctgcgagcacaggtgacagaactggaggatgagctgacagcggccgaggatgccaagctgcgtctggaggtgactgtgcaggctctcaagactcagcatgagcgtgacctgcagggccgtgatgaggctggtgaagagaggcggaggcagctggccaagcagctgagagatgcagaggtggagcgggatgaggagcggaagcagcgcactctggccgtggctgcccgcaagaagctggagggagagctggaggagctgaaggctcagatggcctctgccggccagggcaaggaggaggcggtgaagcagcttcgcaagatgcaggcccagatgaaggagctatggcgggaggtggaggagacacgcacctcccgggaggagatcttctcccagaatcgggaaagtgaaaagcgcctcaagggcctggaggctgaggtgctgcggctgcaggaggaactggccgcctcggaccgtgctcggcggcaggcccagcaggaccgggatgagatggcagatgaggtggccaatggtaaccttagcaaggcagccattctggaggagaagcgtcagctggaggggcgcctggggcagttggaggaagagctggaggaggagcagagcaactcggagctgctcaatgaccgctaccgcaagctgctcctgcaggtagagtcactgaccacagagctgtcagctgagcgcagtttctcagccaaggcagagagcgggcggcagcagctggaacggcagatccaggagctacggggacgcctgggtgaggaggatgctggggcccgtgcccgccacaagatgaccattgctgcccttgagtctaagttggcccaggctgaggagcagctagagcaagagaccagagagcgcatcctctctggaaagctggtgcgcagagctgagaagcggcttaaagaggtggtgctccaggtggaggaggagcggagggtggctgaccagctccgggaccagctggagaagggaaaccttcgagtcaagcagctgaagcggcagctggaggaggccgaggaggaggcatcccgggctcaggccggccgccggaggctgcagcgtgagctggaagatgtcacagagtcggccgagtccatgaaccgtgaagtgaccacactgaggaaccggcttcgacgcggccccctcaccttcaccacccgcacggtgcgccaggtcttccgactagaggagggcgtggcatccgacgaggaggcagaggaagcacagcctgggtctgggccatccccggagcctgaggggtccccaccagcccacccccagtgaccctaccctgtccccagatgcactaacagatggggcccagcccccttcctccctggaccccacgggcccctgtcccaggaaccccgccctctgacttcttgccctttggaaatggtgcagcactctggcatttatcacccccacctgggtcccctgcaacctcccatcaaaggatgacccctaaacacagaggagcggggcaggcagggaggcaatgactggagctaccttgcttgttgggggactgggtacagttggcaagctgtgtttccatcagctccctgtcctcctttcttccctcgttattgatctatagacattaggaagggagtgagacggctcctccaccatcctcagccagtgcaacccattccctctgcttctctctctctctctctctctccctccctctccttccctaccctctcaccatctttcttggcctctctgagggtctctctgtgcatctttttaggaatctcgctctcactctctacgtagccactctccttcccccatttctgcgtccacccctgaactcctgagcgacagaagccccaggcctccaccagccttgaacccttgcaaaggggcaggacaaggggacccctctcactcctgctgctgcccatgctctgccctcccttctggttgctctgagggttcggagcttccctctgggactaaaggagtgtcctttaccctcccagcctccaggctctggcagaaataaactccaacccgactggaccataaaaaaaaaaaaaaaaaa

SEQ ID NO: 2 - protein, 2003 aa, Homo sapiens
  mat_peptide (1)..(2003): Myh14 protein
MAAVTMSVPGRKAPPRPGPVPEAAQPFLFTPRGPSAGGGPGSGTSPQVEWTARRLVWVPSELHGFEAAALRDEGEEEAEVELAESGRRLRLPRDQIQRMNPPKFSKAEDMAELTCLNEASVLHNLRERYYSGLIYTYSGLFCVVINPYKQLPIYTEAIVEMYRGKKRHEVPPHVYAVTEGAYRSMLQDREDQSILCTGESGAGKTENTKKVIQYLAHVASSPKGRKEPGVPASVSTVSYGELERQLLQANPILEAFGNAKTVKNDNSSRFGKFIRINFDVAGYIVGANIETYLLEKSRAIRQAKDECSFHIFYQLLGGAGEQLKADLLLEPCSHYRFLTNGPSSSPGQERELFQETLESLRVLGFSHEEIISMLRMVSAVLQFGNIALKRERNTDQATMPDNTAAQKLCRLLGLGVTDFSRALLTPRIKVGRDYVQKAQTKEQADFALEALAKATYERLFRWLVLRLNRALDRSPRQGASFLGILDIAGFEIFQLNSFEQLCINYTNEKLQQLFNHTMFVLEQEEYQREGIPWTFLDFGLDLQPCIDLIERPANPPGLLALLDEECWFPKATDKSFVEKVAQEQGGHPKFQRPRHLRDQADFSVLHYAGKVDYKANEWLMKNMDPLNDNVAALLHQSTDRLTAEIWKDVEGIVGLEQVSSLGDGPPGGRPRRGMFRTVGQLYKESLSRLMATLSNTNPSFVRCIVPNHEKRAGKLEPRLVLDQLRCNGVLEGIRICRQGFPNRILFQEFRQRYEILTPNAIPKGFMDGKQACEKMIQALELDPNLYRVGQSKIFFRAGVLAQLEEERDLKVTDIIVSFQAAARGYLARRAFQKRQQQQSALRVMQRNCAAYLKLRHWQWWRLFTKVKPLLQVTRQDEVLQARAQELQKVQELQQQSAREVGELQGRVAQLEEERARLAEQLRAEAELCAEAEETRGRLAARKQELELVVSELEARVGEEEECSRQMQTEKKRLQQHIQELEAHLEAEEGARQKLQLEKVTTEAKMKKFEEDLLLLEDQNSKLSKERKLLEDRLAEFSSQAAEEEEKVKSLNKLRLKYEATIADMEDRLRKEEKGRQELEKLKRRLDGESSELQEQMVEQQQRAEELRAQLGRKEEELQAALARAEDEGGARAQLLKSLREAQAALAEAQEDLESERVARTKAEKQRRDLGEELEALRGELEDTLDSTNAQQELRSKREQEVTELKKTLEEETRIHEAAVQELRQRHGQALGELAEQLEQARRGKGAWEKTRLALEAEVSELRAELSSLQTARQEGEQRRRRLELQLQEVQGRAGDGERARAEAAEKLQRAQAELENVSGALNEAESKTIRLSKELSSTEAQLHDAQELLQEETRAKLALGSRVRAMEAEAAGLREQLEEEAAARERAGRELQTAQAQLSEWRRRQEEEAGALEAGEEARRRAAREAEALTQRLAEKTETVDRLERGRRRLQQELDDATMDLEQQRQLVSTLEKKQRKFDQLLAEEKAAVLRAVEERERAEAEGREREARALSLTRALEEEQEAREELERQNRALRAELEALLSSKDDVGKSVHELERACRVAEQAANDLRAQVTELEDELTAAEDAKLRLEVTVQALKTQHERDLQGRDEAGEERRRQLAKQLRDAEVERDEERKQRTLAVAARKKLEGELEELKAQMASAGQGKEEAVKQLRKMQAQMKELWREVEETRTSREEIFSQNRESEKRLKGLEAEVLRLQEELAASDRARRQAQQDRDEMADEVANGNLSKAAILEEKRQLEGRLGQLEEELEEEQSNSELLNDRYRKLLLQVESLTTELSAERSFSAKAESGRQQLERQIQELRGRLGEEDAGARARHKMTIAALESKLAQAEEQLEQETRERILSGKLVRRAEKRLKEVVLQVEEERRVADQLRDQLEKGNLRVKQLKRQLEEAEEEASRAQAGRRRLQRELEDVTESAESMNREVTTLRNRLRRGPLTFTTRTVRQVFRLEEGVASDEEAEEAQPGSGPSPEPEGSPPAHPQ

SEQ ID NO: 3 - DNA, 25 bp, Artificial Sequence
  P AF primer
agagtagactgtagggagagcaagg

SEQ ID NO: 4 - DNA, 24 bp, Artificial Sequence
  P AR primer
agagggtgttaattgcagaaagtc

SEQ ID NO: 5 - DNA, 22 bp, Artificial Sequence
  P BF primer
cttccaaccttgggaagtcttt

SEQ ID NO: 6 - DNA, 21 bp, Artificial Sequence
  P BR primer
atgaatgggggcctttgtaag

SEQ ID NO: 7 - DNA, 25 bp, Artificial Sequence
  P CF primer
gtacatggtctacgttcgacaaaag

SEQ ID NO: 8 - DNA, 20 bp, Artificial Sequence
  P CR primer
agagagcagcagaggccaat

SEQ ID NO: 9 - DNA, 20 bp, Artificial Sequence
  N1F primer
attggcctctgctgctctct

SEQ ID NO: 10 - DNA, 19 bp, Artificial Sequence
  N1R primer
aaggtgagtgtccgcgtca

SEQ ID NO: 11 - DNA, 24 bp, Artificial Sequence
  2F primer
gaatgaaatgagtaagctgggtct

SEQ ID NO: 12 - DNA, 25 bp, Artificial Sequence
  2R primer
ggatacagatgataaacagcctcaa

SEQ ID NO: 13 - DNA, 25 bp, Artificial Sequence
  3F primer
gttatggtgtagacataccatgtgc

SEQ ID NO: 14 - DNA, 25 bp, Artificial Sequence
  3R primer
aagtctacaagctgtcatttgacct

SEQ ID NO: 15 - DNA, 25 bp, Artificial Sequence
  4-5F primer
gttacacatcaagacccaagctttt

SEQ ID NO: 16 - DNA, 20 bp, Artificial Sequence
  4-5R primer
acctccatgaggggaagaga

SEQ ID NO: 17 - DNA, 23 bp, Artificial Sequence
  6F primer
atcactggttcacctgtgtgtct

SEQ ID NO: 18 - DNA, 23 bp, Artificial Sequence
  6R primer
cctgtctaccaagaagatcatgc

SEQ ID NO: 19 - DNA, 24 bp, Artificial Sequence
  7F primer
atactcagtcagctggagacacag

SEQ ID NO: 20 - DNA, 18 bp, Artificial Sequence
  7R primer
cccctcctccctcaacag

SEQ ID NO: 21 - DNA, 20 bp, Artificial Sequence
  8F primer
gggtttgggctgttgttcac

SEQ ID NO: 22 - DNA, 21 bp, Artificial Sequence
  8R primer
agtgggaggtgctttccatac

SEQ ID NO: 23 - DNA, 23 bp, Artificial Sequence
  9F primer
actccactacaccacaggagaga

SEQ ID NO: 24 - DNA, 20 bp, Artificial Sequence
  9R primer
tcttgcttcctccccagaag

SEQ ID NO: 25 - DNA, 22 bp, Artificial Sequence
  10F primer
gatgaatccaggatgagtctga

SEQ ID NO: 26 - DNA, 20 bp, Artificial Sequence
  10R primer
aaagagatcggggcatgaat

SEQ ID NO: 27 - DNA, 25 bp, Artificial Sequence
  11F primer
aggggtggtgatataatttgcctta

SEQ ID NO: 28 - DNA, 21 bp, Artificial Sequence
  11R primer
atgctctctacgtgggacagg

SEQ ID NO: 29 - DNA, 20 bp, Artificial Sequence
  12F primer
aaacggtaccctctcccttg

SEQ ID NO: 30 - DNA, 22 bp, Artificial Sequence
  12R primer
ggtgggaaaaacagcatacact

SEQ ID NO: 31 - DNA, 25 bp, Artificial Sequence
  13F primer
gcattgttcttgatggtacttacac

SEQ ID NO: 32 - DNA, 20 bp, Artificial Sequence
  13R primer
aggaccggagtgaggaagtt

SEQ ID NO: 33 - DNA, 20 bp, Artificial Sequence
  N14F primer
ctctccccttctccctggtc

SEQ ID NO: 34 - DNA, 24 bp, Artificial Sequence
  N14R primer
gtgagagctctgattaaccgattg

SEQ ID NO: 35 - DNA, 21 bp, Artificial Sequence
  15F primer
tagagtcgggggtttcactgt

SEQ ID NO: 36 - DNA, 21 bp, Artificial Sequence
  15R primer
ccatctgtagccagtggagat

SEQ ID NO: 37 - DNA, 22 bp, Artificial Sequence
  16F primer
gaaggctccttaggaaatccag

SEQ ID NO: 38 - DNA, 23 bp, Artificial Sequence
  16R primer
cagaaaactcaggttcagacctc

SEQ ID NO: 39 - DNA, 25 bp, Artificial Sequence
  17F primer
gatatctaccttacaggctgtgtga

SEQ ID NO: 40 - DNA, 25 bp, Artificial Sequence
  17R primer
ctgagcctaggaatagaagcaattt

SEQ ID NO: 41 - DNA, 20 bp, Artificial Sequence
  18F primer
gaaacaggaaattgccaagc

SEQ ID NO: 42 - DNA, 21 bp, Artificial Sequence
  18R primer
agagtgggtggagacctgaat

SEQ ID NO: 43 - DNA, 25 bp, Artificial Sequence
  19F primer
cttgttattgtcactgttgttcctg

SEQ ID NO: 44 - DNA, 24 bp, Artificial Sequence
  19R primer
aggtaatgaggaaggtcaatgaag

SEQ ID NO: 45 - DNA, 22 bp, Artificial Sequence
  N20F3 primer
cccattactccccctcctcacc

SEQ ID NO: 46 - DNA, 23 bp, Artificial Sequence
  20R primer
acacctagagccatctggtcaac

SEQ ID NO: 47 - DNA, 21 bp, Artificial Sequence
  21F primer
agcttggctctcttgctaagg

SEQ ID NO: 48 - DNA, 22 bp, Artificial Sequence
  21R primer
aaggccctatcctctccctact

SEQ ID NO: 49 - DNA, 23 bp, Artificial Sequence
  22F primer
cagctaataggtggaggagaagg

SEQ ID NO: 50 - DNA, 23 bp, Artificial Sequence
  22R primer
gcctcagtacaccttagctttgc

SEQ ID NO: 51 - DNA, 25 bp, Artificial Sequence
  23F primer
gaacttaaaggccaaagcaagttac

SEQ ID NO: 52 - DNA, 23 bp, Artificial Sequence
  23R primer
acacacggtttgtagaagcaaag

SEQ ID NO: 53 - DNA, 20 bp, Artificial Sequence
  24F primer
ttccccatcacactccatct

SEQ ID NO: 54 - DNA, 20 bp, Artificial Sequence
  24R primer
gagaaccgcagtgtggtaac

SEQ ID NO: 55 - DNA, 22 bp, Artificial Sequence
  25F primer
agatgagaacaacaccagaagc

SEQ ID NO: 56 - DNA, 20 bp, Artificial Sequence
  25R primer
actgaggccctgtagcacat

SEQ ID NO: 57 - DNA, 25 bp, Artificial Sequence
  26F primer
tgagcttagccttatatgtgactgg

SEQ ID NO: 58 - DNA, 20 bp, Artificial Sequence
  26R primer
ctgtctgtggctggtgagtg

SEQ ID NO: 59 - DNA, 25 bp, Artificial Sequence
  27F primer
agagagaggattctaacctgggact

SEQ ID NO: 60 - DNA, 20 bp, Artificial Sequence
  27R primer
acctagagccggtggttcat

SEQ ID NO: 61 - DNA, 24 bp, Artificial Sequence
  28F primer
gaggccctcatattttaaggaaac

SEQ ID NO: 62 - DNA, 24 bp, Artificial Sequence
  28R primer
ggagatgagctagcctagaaccag

SEQ ID NO: 63 - DNA, 23 bp, Artificial Sequence
  N29F primer
gagtgaggggtgagctatttgtt

SEQ ID NO: 64 - DNA, 25 bp, Artificial Sequence
  N29R primer
ctgtgcccagacttgtacatgatta

SEQ ID NO: 65 - DNA, 23 bp, Artificial Sequence
  30-31F primer
gtgtggtagagagttgaggcaga

SEQ ID NO: 66 - DNA, 23 bp, Artificial Sequence
  30-31R primer
gtaagggggagtcagagatgaga

SEQ ID NO: 67 - DNA, 23 bp, Artificial Sequence
  32F primer
cagcttacaccttggtcactcat

SEQ ID NO: 68 - DNA, 23 bp, Artificial Sequence
  32R primer
gattactggttcctcacaacgac

SEQ ID NO: 69 - DNA, 22 bp, Artificial Sequence
  33F primer
cttgtgtctctgcctttgtctg

SEQ ID NO: 70 - DNA, 21 bp, Artificial Sequence
  33R primer
catgtacgtgtctcccctcac

SEQ ID NO: 71 - DNA, 22 bp, Artificial Sequence
  N34F primer
actaggatgggcacactgactt

SEQ ID NO: 72 - DNA, 21 bp, Artificial Sequence
  N34R primer
cctgtacacaccatggcatac

SEQ ID NO: 73 - DNA, 25 bp, Artificial Sequence
  35F primer
gaccaagtaaagagagtcagggagt

SEQ ID NO: 74 - DNA, 22 bp, Artificial Sequence
  35R primer
agctagccttagaccctggagt

SEQ ID NO: 75 - DNA, 21 bp, Artificial Sequence
  36F primer
gagagttccctgcttgttcac

SEQ ID NO: 76 - DNA, 22 bp, Artificial Sequence
  36R primer
acatggtaagacacacccacct

SEQ ID NO: 77 - DNA, 20 bp, Artificial Sequence
  37F primer
ttacccagggacagcatgag

SEQ ID NO: 78 - DNA, 21 bp, Artificial Sequence
  37R primer
ccttgcctcacactacaggac

SEQ ID NO: 79 - DNA, 21 bp, Artificial Sequence
  38F primer
acagggtcctgtagtgtgagg

SEQ ID NO: 80 - DNA, 25 bp, Artificial Sequence
  38R primer
ctattcaaccttcaaaacccaactc

SEQ ID NO: 81 - DNA, 24 bp, Artificial Sequence
  39F primer
cagtgcacttaattctcagaggtg

SEQ ID NO: 82 - DNA, 25 bp, Artificial Sequence
  39R primer
ctaaaccacgtgtttgtaacacagc

SEQ ID NO: 83 - DNA, 25 bp, Artificial Sequence
  40F primer
atgagtctatggggacaaaatccta

SEQ ID NO: 84 - DNA, 21 bp, Artificial Sequence
  40R primer
ctgtgcaatttctcagccagt

SEQ ID NO: 85 - DNA, 20 bp, Artificial Sequence
  41F primer
gatggggcagagagagtcag

SEQ ID NO: 86 - DNA, 20 bp, Artificial Sequence
  41R primer
atcccatttccccctgttgt

SEQ ID NO: 87 - DNA, 24 bp, Artificial Sequence
  42F primer
cccttattttgttctctctcttcc

SEQ ID NO: 88 - DNA, 21 bp, Artificial Sequence
  42R primer
atttccaaagggcaagaagtc

SEQ ID NO: 89 - DNA, 22 bp, Artificial Sequence
  3UTR AF primer
ccaggtcttccgactagaggag

SEQ ID NO: 90 - DNA, 24 bp, Artificial Sequence
  3UTR AR primer
tcctaaaaagatgcacagagagac

SEQ ID NO: 91 - DNA, 21 bp, Artificial Sequence
  3UTR BF primer
cattccctctgcttctctctc

SEQ ID NO: 92 - DNA, 22 bp, Artificial Sequence
  3UTR BR primer
caggtgtcattctaaccagcag